(19) United States
(12) Patent Application Publication
(10) Pub. No.: US 2025/0255736 A1
(43) Pub. Date: Aug. 14, 2025

(54) Sensing System for Monitoring Prosthetic Socket Fit

(71) Applicant: University of Washington, Seattle, WA (US)

(72) Inventors: Joan E. Sanders, Seattle, WA (US); Joseph Mertens, Seattle, WA (US); Nicholas DeGrasse, Seattle, WA (US); Katheryn J. Allyn, Seattle, WA (US); Ryan Carter, Seattle, WA (US); Adam Krout, Seattle, WA (US); Brian Hafner, Seattle, WA (US); Conor Lanahan, Seattle, WA (US)

(21) Appl. No.: 18/867,101

(22) PCT Filed: Jun. 2, 2023
(86) PCT No.: PCT/US2023/067819

§ 371 Date: Nov. 19, 2024

Related U.S. Application Data
(60) Provisional application No. 63/348,430, filed on Jun. 2, 2022.

(51) Int. Cl. A61F 2/76; A61F 2/50; A61F 2/60; A61F 2/70; A61F 2/78

(52) U.S. Cl. CPC A61F 2/76; A61F 2/5046; A61F 2/60; A61F 2/70; A61F 2/7812; A61F 2002/5053; A61F 2002/608; A61F 2002/704; A61F 2002/762

(57) ABSTRACT

The present disclosure provides methods for fabricating an instrumented prosthetic socket including a. layered, fabrication with inductive sensors positioned between electrically insulating layers of the socket. The present disclosure further provides methods for tracking of position relationship between socket and socket liner using inductive sensor arrays, and inductive sensor configurations for placement on a curved surface.

### CROSS-REFERENCE TO RELATED APPLICATIONS

This application claims the benefit of the filing date of U.S. Provisional Patent Application Ser. No. 63/348,430, filed Jun. 2, 2022, which is hereby incorporated by reference in its entirety.

### FEDERAL FUNDING

This invention was made with government support under Grant No. W81XWH-16-C-0020, awarded by the Department of Defense and Grant No. R01HD60585, awarded by the National Institutes of Health (NIH). The government has certain rights in the invention.

### BACKGROUND

Unless otherwise indicated herein, the materials described in this section are not prior art to the claims in this application and are not admitted to be prior art by inclusion in this section.

Distance sensing offers advantages over the traditional technique of pressure sensing for monitoring limb-socket interactions in people using transtibial prostheses. Distance sensing is not limited by contact with the sensor and captures elements of swing phase, not just stance phase, allowing a more complete characterization of how people use their prosthesis. Pressure sensing data can be less precise because of its relatively low resolution. sensitivity to crosstalk from other directions of stress, and the socket shape distortion and material property discontinuities its sensing element introduces. Distance sensing also allows monitoring of limb volume and socket fit within a range of comfort for a prosthetic user, and with an active prosthetic fit adjustment system could provide management of limb volume and socket comfort over the course of prosthetic use, though no system is currently offered that reliably incorporates distance sensing into a prosthetic socket.

### SUMMARY

The present disclosure provides methods to embed distance sensors within prosthetic sockets. In particular, the present disclosure describes a layered socket with embedded distance sensors placed in a fixed, curved position. The distance sensors may be configured for measuring socket fit and/or for monitoring prosthesis use. The present disclosure further provides a trilateralized distance sensor setup to monitor distance and sensor target translation.

Thus, in a first aspect, the present disclosure provides a prosthetic socket comprising: (a) a first layer defining a cavity, a second layer positioned over the first layer, (c) a plurality of sensors positioned between the first layer and the second laver, wherein each of the plurality of sensors are configured to measure a distance from a residual limb positioned within the cavity, and (d) a communication interface in communication with each of the plurality of sensors.

In one example of the first aspect. each of the plurality of sensors is an inductive sensor.

In another example of the first aspect, the first layer further comprises an electrically insulating webbing material and a curable matrix.

In another example of the first aspect, the second layer further comprises carbon fiber webbing and a curable matrix.

In another example of the first aspect, each of the plurality of sensors has a ground wire threaded into carbon fiber layer.

In another example of the first aspect, each of the plurality of sensors further comprises: (a) an antenna, (b) a thermistor, and (c) a capacitor, wherein the thermistor and the capacitor are co-located with the antenna.

In another example of the first aspect, each of the plurality of sensors further comprises: (a) a flexible antenna having a front side and a back side, (b) an electrically insulating layer having a front side and a back side, wherein the front side is fixed to the back side of the flexible antenna, and (c) a ferrite outer layer fixed to the back side of the electrically insulating layer.

In another example of the first aspect, the inductive sensor further comprises a looped antenna fixed to a flexible substrate, and wherein the flexible substrate includes a cut out substantially in the center of a circle defined by the looped antenna.

In another example of the first aspect, the sensor layers have an outer perimeter edge and an inner perimeter edge defined by the cut out, and wherein any of the sensor lavers include alternating radial slit cuts from the outer perimeter toward the looped antenna and from the inner perimeter toward the looped antenna.

In another example of the first aspect, the plurality of sensors are configured to measure socket fit during prosthesis use.

In another example of the first aspect, a first sensor is positioned in a posterior distal region of the socket at a first distance from a midline of the socket, a second sensor is positioned in a posterior distal region of the socket at the first distance from the midline opposite the first sensor, a third sensor positioned in an anterior medial proximal region of the socket, and a fourth sensor in an anterior lateral proximal region of the socket.

In another example of the first aspect, the plurality of sensors is configured to measure socket fit to provide feedback for adjusting socket fit.

In another example of the first aspect, a first sensor is positioned at an anterior distal end of the socket, a second sensor is positioned at an anterior proximal end of the socket, a third sensor is positioned between sensor one and sensor two on an anterior side of the socket, a fourth sensor is positioned at a medial mid-limb location on a posterior side of the socket, a fifth sensor is positioned at a lateral mid-limb location on a posterior side of the pocket, and a sixth sensor is positioned at a posterior distal end of the socket.

In another example of the first aspect, the prosthetic socket further comprises: (a) a controller configured to receive a set of signals from the plurality of sensors, (b) an adjustable panel in the socket configured to loosen or tighten socket fit with adjustment, (c) an electric motor configured to move the adjustable panel, and (d) a power source, wherein the controller is configured to actuate the electric motor based on, at least in part, received signals from the plurality of sensors.

In another example of the first aspect, the controller actuates the adjustable panel to loosen in response to the set of signals from the plurality of sensors reporting increasing limb volume, and the controller actuates the adjustable panel to tighten in response to the set of signals from the plurality of sensors reporting decreasing limb volume, wherein the controller sets a comfortable fit distance from the plurality of sensors upon donning the socket, and the controller works to maintain the comfortable fit distance.

In another example of the first aspect, the set of received signals from the plurality of sensors classify one of the following prosthetic use conditions: (a) stationary sitting, (b) stationary standing, (c) sit-to-stand transitioning, (d) walking, (e) weight-shifting sitting, (f) weight-shifting standing, (g) partial doffing, and (h) non-use.

In a second aspect, the present disclosure provides a method of making an instrumented prosthetic socket comprising: (a) forming an inner layer of material over a mold, (b) placing a plurality of sensors on an outer surface of the inner layer, and (c) forming an outer layer of material over the sensors and inner layer.

In one example of the second aspect, the inner layer comprises an electrically insulating webbing and a curing matrix.

In another example of the second aspect, placing a plurality of sensors includes placing wiring from the sensors to a controller.

In a third aspect, the present disclosure provides a prosthetic socket comprising: (a) a socket configured to receive a limb, wherein the socket further comprises: (i) a first layer, and (ii) a second layer, and (b) a plurality of sensor arrays between the first layer and second layer of the socket, wherein each sensor array of the plurality of sensor arrays comprises at least three sensors, and wherein each sensor array is configured to measure a three-dimensional position from the sensor array's position in the socket to a single target material.

In one example of the third aspect, each sensor is configured to provide to a controller a distance data to a sensor target and wherein the controller trilateralizes the distance data from each sensor array to provide a three dimensional position data of the sensor target relative to the sensor array.

In another example of the third aspect, a lookup table converts the distance data from each of the at least three sensors to a three dimensional position data.

### BRIEF SUMMARY OF THE DRAWINGS

FIG. 1A illustrates a side view of prosthetic socket, according to an example embodiment.

FIG. 1B illustrates a top view of the prosthetic socket of FIG. 1A, according to an example embodiment.

FIG. 2A illustrates a sensor before placement in the prosthetic socket, according to an example embodiment.

FIG. 2B illustrates a sensor with ferrite backing layer, according to an example embodiment.

FIG. 2C illustrates a sensor with radial slits cut into ferrite backing layer to improve flexibility, according to an example embodiment.

FIG. 3A illustrates a sensor configuration for prosthesis use monitoring, according to an example embodiment.

FIG. 3B illustrates a configuration for socket fit feedback, according to an example embodiment.

FIG. 4 illustrates a cross-section of layers from socket wall with embedded sensor through to limb sock with target material for sensing distance. according to an example embodiment.

FIG. 5 is a flow chart illustrating steps used to classify sensed distance data into categories of prosthesis use, according to an example embodiment.

FIG. 6 is a flow chart illustrating steps for socket fabrication with embedded sensors, according to an example embodiment.

FIG. 7A illustrates a sensor array for trilateration, according to an example embodiment.

FIG. 7B illustrates distance data gathered from trilateration sensor array during walking, according to an example embodiment.

FIG. 7C illustrates a target material installed into socket liner to ensure the trilateration sensor array provides information relevant to the location of the target rather than each providing shortest path distance measurements, according to an example embodiment.

FIG. 8 illustrates a limb volume management algorithm for a socket with a motorized adjustable panel, according to an example embodiment.

FIG. 9 illustrates a walking cycle plan for distance sensor data gathering, according to an example embodiment.

FIG. 10 illustrates a panel position data during cycles of activity and limb volume changes in a prosthesis using the socket fit configuration of sensors and a motorized panel adjustment system, according to an example embodiment.

FIG. 11 illustrates limb volume data during cycles of activity for different limb volume management strategies, according to an example embodiment.

FIG. 12 illustrates relative reported socket fit data while socket fit adjustments being made based on distance sensor data while limb volume management system is employed, according to an example embodiment.

FIG. 13 illustrates a 3D printed plastic insert for the bottom of a prosthesis socket having recessed positions for distance sensors to be placed, according to an example embodiment.

FIG. 14 illustrates a plastic insert for bottom of prosthesis socket with sensors installed in position, according to an example embodiment.

### DETAILED DESCRIPTION

Example devices, methods, and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As used herein, with respect to measurements, “about” means +/−5%.

The present disclosure provides description of devices including sockets for a prosthetic limb having distance sensors embedded within the socket and the methods of making such sockets. Distance sensors used in combination with sensor targets in a prosthetic liner or limb sock worn by the user of the prosthesis can measure and monitor the distance between the sensor embedded in the socket and the sensor target in the liner or sock. Measuring and monitoring the distance between the sensor and sensor target at various locations around the limb and socket help track limb volume changes and socket fit, as the prosthetic liner is a tight-fitting garment and a very close proxy to the skin surface of the residual limb. Placing sensors in different locations around the socket can give better socket fit measurements or better prosthesis use monitoring information.

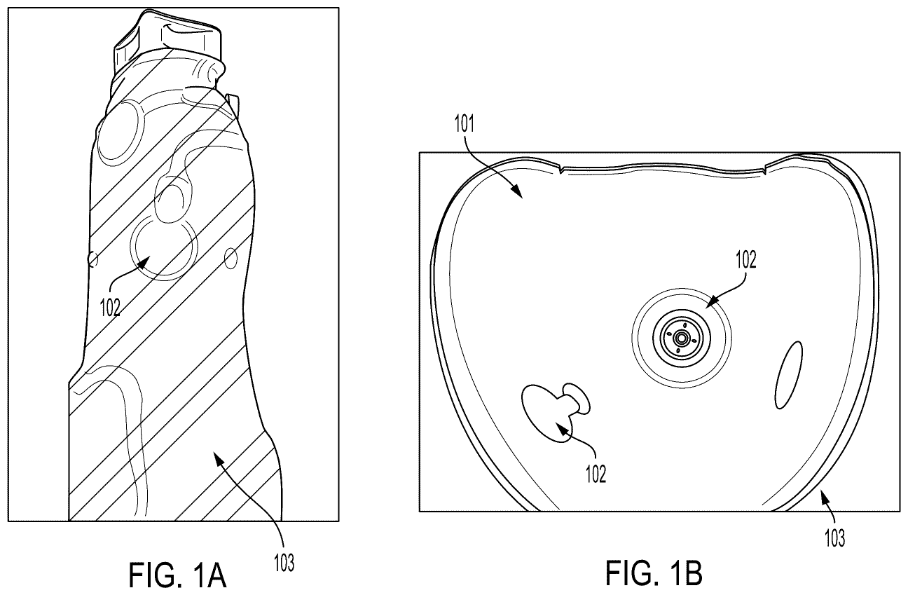

With reference to the Figures, FIG. 1A and 1B show an embodiment of the prosthetic socket. FIG. 1A shows the cavity configured to receive a residual limb, with the inner layer 101 of the prosthetic socket defining the cavity. Within the space defined between the inner layer 101 and the outer layer 103 of the prosthetic socket, distance sensors 102 are placed. The inner layer 101 is an electrically insulating material in preferred embodiments. In demonstrative embodiments described as examples in this detailed description, the electrically insulating material is made from a webbing of Nyglass combined with a curable matrix, such as resin. The outer layer 103 can be a strong, lightweight material, such as a webbing of carbon fiber, and a curable matrix, such as resin.

The socket of a prosthesis is a firm receptacle for the limb to which the prosthesis is secured. A socket will generally include a cavity for the limb to occupy during the donning of the prosthesis.

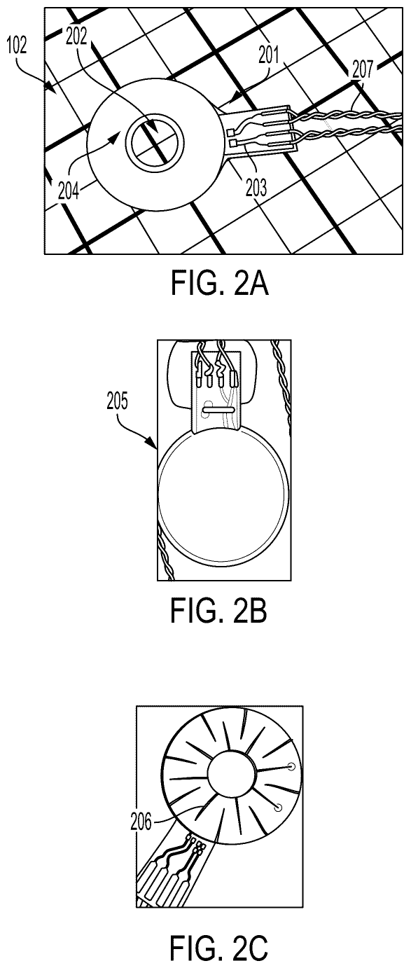

FIGS. 2A-C show embodiments of the sensor 102. FIG. 2A shows a wired sensor with an electrically insulating layer fixed to one side of the sensor substrate and a hole cut from the center of the sensor substrate and electrically insulating layer 201. A thermistor and a capacitor 203 are co-located with each sensor 102 to reduce the opportunity to introduce noise. The composition of each sensor 102 includes the antenna 204, in some embodiments a Nyglass layer on one side (as shown by 201), and then a shielding layer 205 on the side of the Nyglass layer 201 opposite the sensor 102 to prevent electrical interference from outside sources, from the outer carbon fiber layer 103, and/or from other sensors 102 nearby. Another embodiment includes the antenna 204, the shielding layer 205, and then a Nyglass layer 201 on top of the shielding layer 205. The shielding layer 205 in the representative embodiment was ferrite. FIG. 2C shows a ferrite shielding layer 205 with radial slits 206 cut in an alternating pattern from the inner perimeter of the center hole 202 and the outer perimeter of the sensor 102. The slits 206 allow for greater flexibility of the sensor 102 when placing it on a curved surface, such as in a smaller socket for a child.

As shown in FIG. 2A, the sensor 102 further includes a communication interface 207 in communication the sensor 102. The communication interface 207 enables transmission of data from the sensor 102 to an external device for further analysis and processing. The external device may be any type of device that can receive data and display information corresponding to or associated with the data. For example, the external device may be a mobile phone, a tablet, or a personal computer as examples. The sensor 102 and the external device may contain hardware to enable the communication interface 207 to operate, such as processors, transmitters, receivers, antennas, etc. In FIG. 2A, the communication interface 207 is illustrated as a wired connection; however, wireless connections may also be used. For example, the communication interface 207 may be a wired link via a serial bus such as a universal serial bus or a parallel bus. A wired connection may be a proprietary connection as well. The communication interface 207 may also be a wireless connection using, e.g., Bluetooth® radio technology, communication protocols described in IEEE 802.11 (including any IEEE 802.11 revisions), Cellular technology (such as GSM, CDMA, UMTS, EV-DO, WiMAX, or LTE), or Zigbee® technology, among other possibilities.

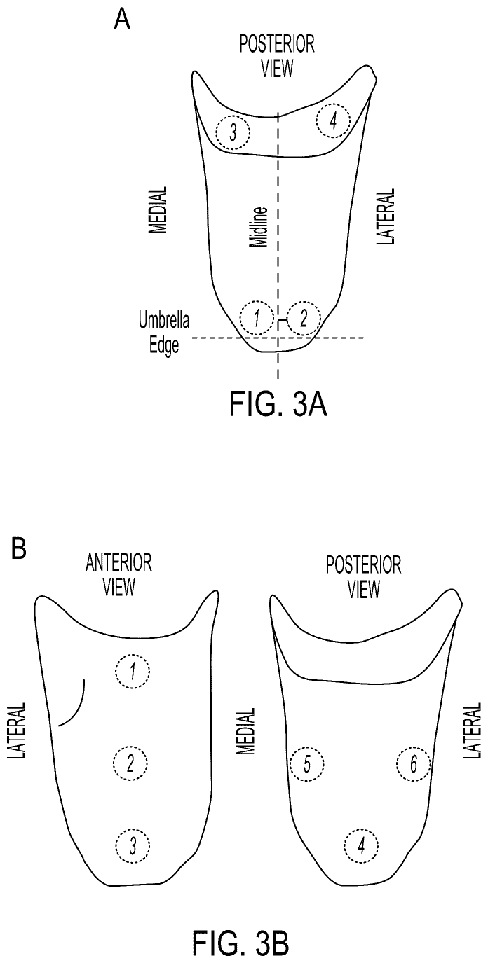

As shown in FIG. 3A, in some embodiments of the prosthesis the sensors are configured to better collect data relevant to delineating activity and bodily position during prosthesis use. This configuration may use as few as two sensors—either a proximal pair (FIG. 3A—items 3 and 4) or a distal pair FIG. 3A—items 1 and 2), but four or more sensors is preferred. Four sensors would be positioned as indicated by the corresponding numbered callouts of FIG. 3A, and as follows: sensor one positioned in a posterior distal region at a first distance from the midline of the socket; sensor two is positioned in a posterior distal region at the first distance from the midline opposite sensor one; sensor three position in an anterior medial proximal region; and sensor four in an anterior lateral proximal region. The prosthesis use configuration of sensors collect data which can classify at least the following use conditions: stationary sits, stationary stands, walks (low locomotion, bouts), weight-shifting (sitting, standing), partial doffs, and non-use (full doffs). Information gathered by the sensors could be received by a controller or datalogger, which may be either in communication via wire or wirelessly with the sensors.

As shown in FIG. 3B, in some embodiments of the prosthesis the sensors are configured to better collect data relevant to measure the socket fit, such as during use of a prosthesis with an auto-adjusting fit. This configuration may use as few as two sensors, such as a distal and mid-limb pair (FIG. 3B—items 4 and 5, 4 and 6, 3 and 5, or 3 and 6), though using 6 or more sensors is preferred. Six sensors would be positioned as indicated in the corresponding numbered callouts of FIG. 3B, and as follows: sensor one is positioned at an anterior distal end of the prosthetic socket; sensor two is positioned at an anterior proximal end of the prosthetic socket; sensor three is positioned between sensor one and sensor two on the anterior side of the prosthetic socket; sensor four is positioned at a medial mid-limb location on a posterior side of the prosthetic socket; sensor five is positioned at a lateral mid-limb location on a posterior side of the prosthetic socket; and sensor six is positioned at a posterior distal end of the prosthetic socket. The socket fit configuration of sensors can provide feedback to a controller which may then drive changes to the socket fit by via an electric motor, powered by a power source, and the electric motor is configured to adjust one or more moveable components interfacing with the limb to tighten or loosen the socket fit. The controller may be connected by wire to the sensors or sensor network, or may be connected wirelessly to the sensors or sensor network.

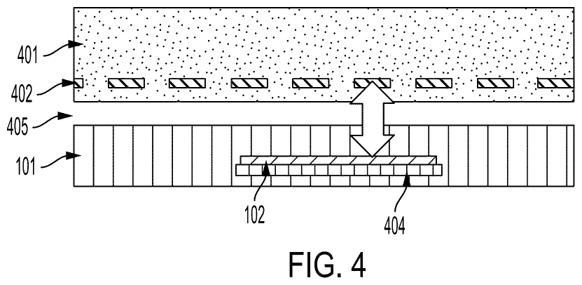

As shown in FIG. 4, in several embodiments of the prosthesis, the sensors 102 used are inductive sensors. The sensor 102 detects a target material 402 in the limb liner or sock 401 and the target material 402 is any suitable material an inductive sensor detects well; in many cases the target material 402 used is magnetic and can be a layer through the entire limb liner or sock, or a patch placed in a known location an inductive sensor is matched to in the socket 101. The target can also be a conductive material. The sensor 102 uses a backing layer 404 of a material to reduce interference to the sensor 102. Part of the distance sensed between the sensor 102 and the target material 402 is an air gap 405 that would increase and decrease depending on how tight the fit of the socket 101 is on the limb and limb sock 401.

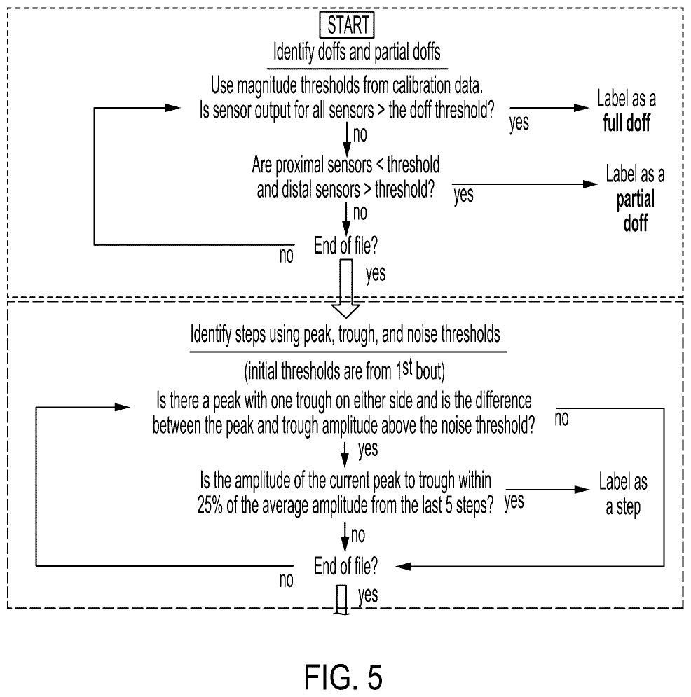

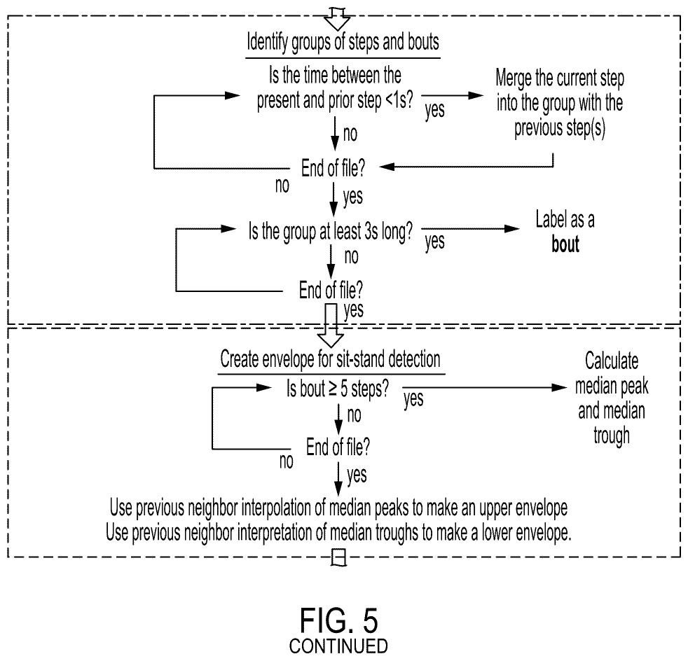

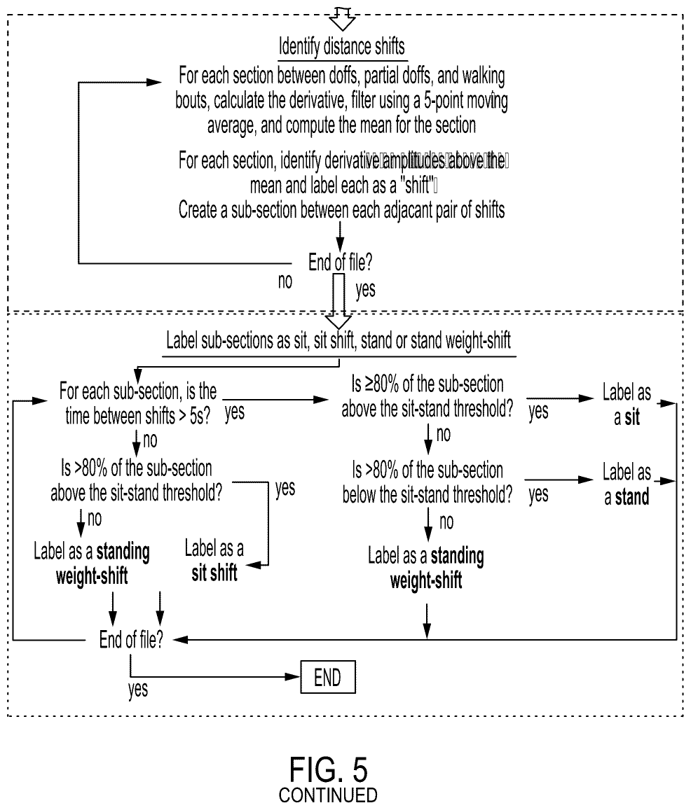

FIG. 5 is a comprehensive chart of the algorithm used to classify prosthesis use activity using distance sensor data. Doffs and partial doffs of at least 5 s duration were identified. A doff threshold was identified for each sensor as the lowest sensed value within a known doff for the scripted and semi-scripted protocols. Since the sensor signal magnitude was so much greater for doffs than dons, minimal error was introduced using this method. If all four sensors were above the doff threshold for longer than 5 s, then the prosthesis was considered doffed. If the two distal sensors were above the doff threshold but the two proximal sensors were not, then the prosthesis was considered partially doffed. If all four sensors were below the doff threshold, then the prosthesis was considered donned. Doffs shorter than 5 s typically were not present in the data. If they had occurred, then they would have been classified as standing weight-shifts.

For the donned sections of data, to identify walking bouts we implemented a method using peak and trough thresholds to identify steps and determine how many steps were in each bout. An initial amplitude (peak minus trough) threshold is selected via visual inspection of the data midway through the first bout of use. This was done to exclude noise oscillations from being picked up as walking. If in the data stream there was a maximum above the peak threshold between two minima below the trough threshold, then the action was considered a step. The algorithm adjusted the threshold peak and trough over time depending on the history of maxima and minima detected. After completion of this process, the algorithm searched through the data stream again to identify pauses between adjacent groups of steps shorter than 1 s. It consolidated the two adjacent groups of steps into one contiguous bout. The time threshold for a walking bout was set to 3 s. Steps not within walking bouts were retained and classified as part of stand shifts as described below.

The walking data were further processed to facilitate identification of sitting and standing bodily positions, the next step in analysis. Participants' sensor stance phase minima and swing phase maxima tended to change over the course of the day. For the expected result of fluctuations in limb volume, these changes needed to be tracked because they set the envelope for detection of sitting and standing positions between bouts, as described below. Plotting the median peaks and median troughs of the last 5 steps in each walking bout and then linearly interpolating between adjacent points creates a continuous envelope. Bouts <5 steps were not included in creating the envelope because excessive step-to-step fluctuation within those bouts, likely due to the residual limb moving down in the socket during these first steps after a doff, was found to occasionally cause incorrect sit and stand identification.

The remaining activities and bodily positions not yet classified took place between the walking bouts and between walking bouts and the times the prosthesis was doffed or partially doffed. To identify sitting and standing within each of these sections, significant sensed distance shifts were first identified. Sensed distance shifts, hereafter termed “shifts,” delineated a change in bodily position. This was done by calculating the derivative from each sensor across the section, filtering that result using a 5-point moving average, and then computing the mean of the absolute value for the section. The mean served as an amplitude threshold for identifying shifts. A strategy that avoids using a derivative calculation is preferred, since derivatives tend to be unstable, and development of a revised strategy is considered a future research objective once more data are available. A result above the threshold was considered a shift. Data between shifts longer than 5 s were labelled as sitting or standing bodily positions using the identification procedure described in the following paragraph. Data between shifts shorter than 5 s were characterized as seated shifts or standing weight-shifts as described below.

To label data between shifts as a sit or a stand, a sit-stand threshold was specified for each participant. The threshold was selected using a confusion matrix and optimization procedure on data from a semi-scripted protocol. A confusion matrix is a technique for assessing the performance of a classification algorithm. The optimization iterated through the data and selected a threshold value that yielded the greatest combined value of sit precision, stand precision, sit sensitivity, and stand sensitivity when compared to the true value from the video data. The thresholds were expressed as a percentage of the envelope from the walking bout analysis described above. If >80% of the sensor data between the shifts was above the sit/stand threshold, then the bodily position was considered a sit. If >80% of the sensor data between the shifts was below the sit/stand threshold, then the bodily position was considered a stand. If neither of these conditions was met, then the bodily position was considered a standing weight-shift as described below.

Data between shifts that were not classified as walking bouts or sitting or standing bodily positions were defined as seated shifts or standing weight-shifts. The data was defined as a seated shift if all of the points were above the sit-stand threshold. Otherwise, the bodily position was defined as a standing weight-shift.

Data from take-home testing were plotted over time and visually inspected to ensure there were no obvious processing errors. Time points for the beginning of the prosthesis day and end of the prosthesis day were selected generally by noting: the beginning of prosthesis day was classified as the first donned period longer than 30 min; if a donned period shorter than 30 min occurred <60 min before the start of the first donned period longer than 30 min, then it was considered the beginning of the prosthesis day. The end of the prosthesis day was defined as the start of a doff period longer than 60 min such that there were no donned periods longer than 30 min between this point and the start of the beginning of the next prosthesis day.

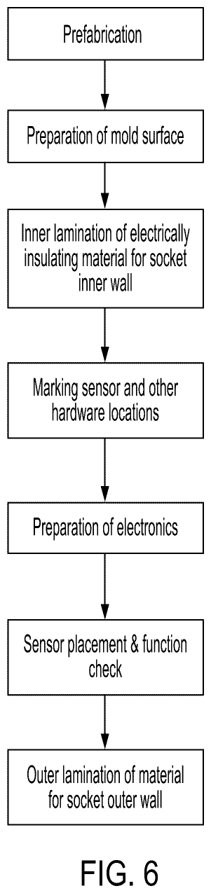

FIG. 6 lays out the steps, generally, to fabricate a prosthesis socket with embedded distance sensors. In several embodiments of the device, sensors are embedded in the inner lining of the socket wall between a first layer or inner layer of the socket wall and a second layer or outer layer of the socket wall. Sensors are placed during the layer-by-layer process of making the socket. A model of the limb the prosthesis is being made to fit is used to build the prosthetic socket. The model need not be a perfect representation of the limb, but a cast representation of the individual limb may be used. In a representative embodiment of socket fabrication, a foam model was used. Over the foam model, one or more layers of an electrically insulating material also suitable for casting to the shape of the limb model are used. In the representative embodiment, two layers of Nyglass and resin are used for this inner lining of the socket wall. Upon curing, drying, and/or setting the inner lining, sensors are placed on the outer surface of the inner lining in a desired configuration such as prosthesis use or socket fit, discussed above.

Following placement of the sensors and associated wiring and other components (ex. controller, data acquisition plug, etc.) a second lamination is completed over the components and adding further strength to the socket. The second lamination can use additional Nyglass in areas where electrical insulation is helpful. such as at the base of the data acquisition plug. Otherwise, carbon fiber is laid out over the socket Electrical components may have ground wires threaded into the electrically conductive carbon fiber layer. Once the resin used with the carbon fiber layer is cured, the socket may be used, though if additional hardware is being installed, it would be placed over the cured carbon fiber layer of the second lamination, and an additional lamination of Nyglass and then another lamination of carbon fiber finishes the socket. Additional hardware may include an adjustable panel for tightening or loosening the socket fit.

In some examples, one or more components of the prosthetic device described above is made via an additive manufacturing process using an additive-manufacturing machine, such as stereolithography, multi-jet modeling, inkjet printing, selective laser sintering/melting, and fused filament fabrication, among other possibilities. Additive manufacturing enables one or more components of the prosthetic device and other physical objects to be created as an interconnected single-piece structure through the use of a layer-upon-layer generation process. Additive manufacturing involves depositing a physical object in one or more selected materials based on a design of the object. For example, additive manufacturing can generate one or more components of the prosthetic device using a Computer Aided Design (CAD) of the prosthesis as instructions. As a result, changes to the design of the prosthetic socket can be immediately carried out in subsequent physical creations of the prosthetic device. This enables the components of the prosthetic socket to be easily adjusted or scaled to fit different types of applications (e.g., for use in various prosthesis sizes).

The layer-upon-layer process utilized in additive manufacturing can deposit one or more components of the prosthetic device with complex designs that might not be possible for devices assembled with traditional manufacturing. In turn, the design of the prosthetic device can include aspects that aim to improve overall operation. For example, the design can incorporate physical elements that help redirect stresses or tissue motion in a desired manner that traditionally manufactured devices might not be able to replicate.

Additive manufacturing also enables depositing one or more components of the prosthetic device in a variety of materials using a multi-material additive-manufacturing process. In such an example, the layers of the prosthetic socket may be made from a first material and the uncured inductive sensors may be made from a second material that is different than the first material. In another example, only the inductive sensors are manufactured using additive manufacturing.

In addition, various processes are used in other examples to produce one or more components of the prosthetic device. These processes are included in table 1.

TABLE 1
Select additive manufacturing methods for
production of laminated prosthetic socket.

| | |
|---|---|
| DEP | Direct Energy Deposition |
| DMLS | Direct Metal Laser Sintering |
| DMP | Direct Metal Printing |
| EBAM | Electron Beam Additive Manufacturing |
| EBM | Electron Beam Melting |
| EBPD | Electron Beam Powder Bed |
| FDM | Fused Deposition Modeling |
| IPD | Indirect Powder Bed |
| LCT | Laser Cladding Technology |
| LDT | Laser Deposition Technology |
| LDW | Laser Deposition Welding |
| LDWM | Laser Deposition Welding with integrated |
| | Milling |
| LENS | Laser Engineering Net Shape |
| LFMT | Laser Freeform Manufacturing Technology |
| LMD-p | Laser Metal Deposition-powder |
| LMD-w | Laser Metal Deposition-wire |
| LPB | Laser Powder Bed |
| LPD | Laser Puddle Deposition |
| LRT | Laser Repair Technology |
| PDED | Powder Directed Energy Deposition |
| SLA | Stereolithography |
| SLM | Selective Laser Melting |
| SLS | Selective Laser Sintering |
| SPD | Small Puddle Deposition |

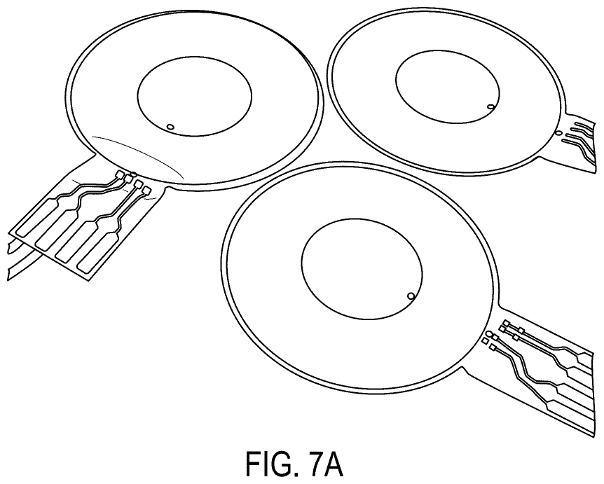

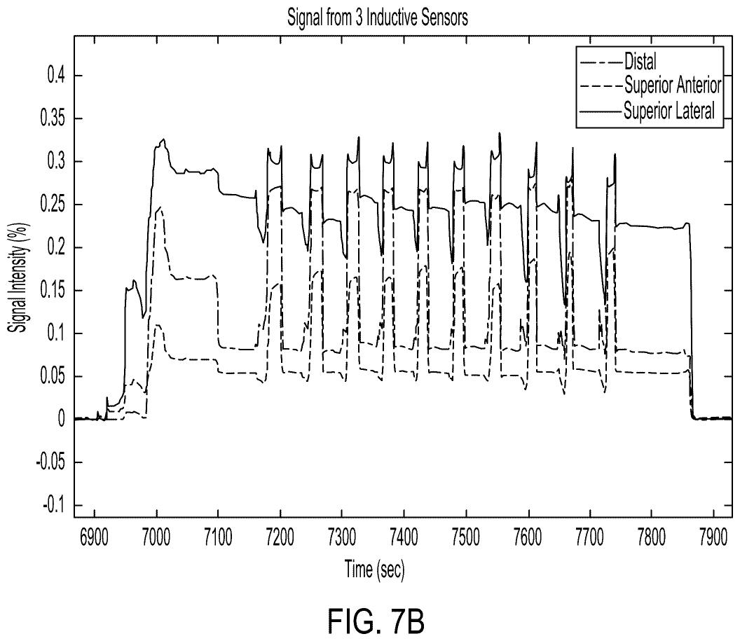

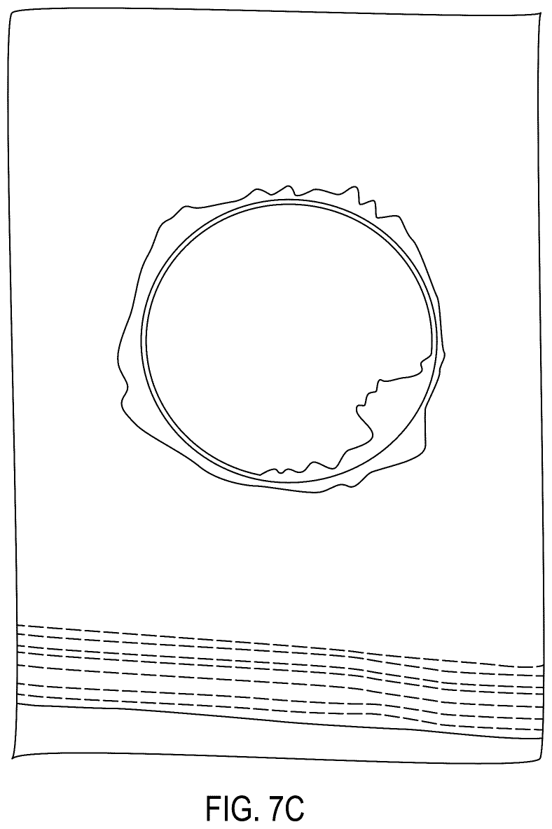

FIG. 7A shows a distance sensor array that is used to trilaterate the distance between the target material in the limb sock and the sensors embedded in the socket wall. A sensor array providing trilateration of the distance measurement offers a finer resolution of the distance detected, is more robust to an individual sensor failure, and can also track lateral movements of a feature (such as a small patch with target material (ferrite) embedded in a layer) that further inform the quality of socket fit.

FIG. 7B shows data collected from a sensor array as in FIG. 7A, and in this example shows data collected during a walking activity. While the distance information from each sensor differs in quantity, each corresponds well to each other in measuring movement during the activity.

FIG. 7C shows a prosthetic liner with a target placed for use with a sensor array as shown in FIG. 7A. While a layer of a magnetic material, such as ferrite, makes for a good target material 402 in other embodiments, when a sensor array is used, to obtain the benefit of trilateration, a smaller sensor target is preferred.

The trilateration array (TRAY) sensor shown in FIG. 7A was fabricated by taking three of the single inductive sensors and placing them in the array so that the centers form an equilateral triangle with side lengths equal to 32 mm in the experimental embodiment shown, though size could be tailored to any measurement of interest. A single thermistor was placed just at the edge of the array to obtain temperature changes.

The trilateration liner target of FIG. 7C was fabricated by taking an off-the-shelf liner and punching 32 mm holes that match the location of the TRAY sensors to be laminated in the prosthetic socket. 32 mm punches are removed from the custom ferrous polymer embedded liners in locations at about the same position as the punches taken from the off-the-shelf liner. The ferrous polymer punches are taken and glued into the off-the-shelf liner holes with Silpoxy adhesive. Care is taken to have a smooth join between the hole and ferrous punch.

It was identified in preliminary studies that if a 32 mm ferrous polymer laden liner punch was moved above a single inductive sensor in three dimensions, the signal intensity would change in a predictable manner symmetric about the center of the sensor. Thus, given a signal reading from the sensor, it was understood that the target position could be mapped along a dome-like-surface above the sensor (essentially, given a signal intensity we know the distance the target is from the sensor, but not the direction). This functionality thus allows us to apply the mathematical method of trilateration to the sensor and target interaction. In turn two methods were developed: the system of equations method, and lookup table solve method.

In the system of equations method. the equations for true trilateration were adapted to fit the inductive sensor's signal-versus-distance shape, which was modeled with paraboloids instead of spheres. The following equations describe the distance the target is from the sensor. while knowing the position of the sensor.

$$z = {h}_{1} ( 1 - \frac{{x}^{2} + {y}^{2}}{{r}_{1}^{2}} ) \qquad Eq . 1$$

$$z = {h}_{2} ( 1 - \frac{{( x - 32 )}^{2} + {y}^{2}}{{r}_{2}^{2}} ) \qquad Eq . 2$$

$$z = {h}_{3} ( 1 - \frac{{( x - 16 )}^{2} + {( y + \sqrt{{32}^{2} - {16}^{2}} )}^{2}}{{r}_{3}^{2}} ) \qquad Eq . 3$$

In these equations h₁, h₂, h₃ correspond to the z-intercepts, r₁, r₂, r₃ correspond to the x/y-intercepts of the modeled sensing region of the sensor for each of the three sensors and, the x- and y-operators describe when the sensor is in the Cartesian coordinate system based on the sensor locations. Given h and r change with the observed signal intensity and once those are defined, the system of equations is solved for x, y, and z—the position of the target.

In practice, this target tracking technology can also be solved using a lookup table. This can be done because for each position the target can be found within the sensing region there will be a unique signal signature when taking the data from all three sensors. Initially the triplets of sensor signal are obtained from a sampling of positions above the TRAY. This lookup table is then used to map the incoming signal triplet to the closest signal triplet found and thus position is determined. In our current application, we are able to linearly interpolate signal triplets to have a finer resolution of position detection than what was run in the initial experiment.

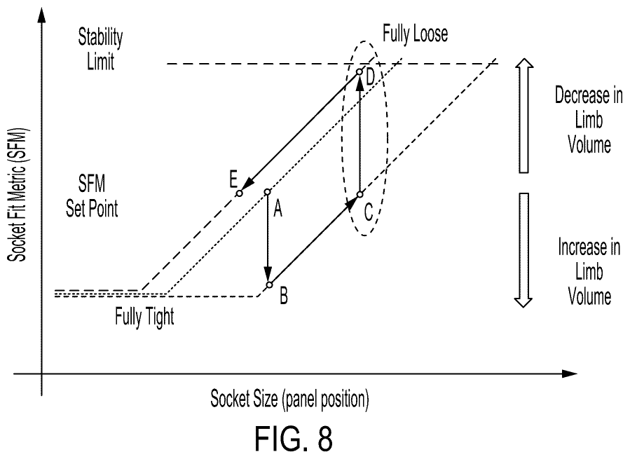

FIG. 8 shows an embodiment of an auto-adjustment algorithm operated during walking. It begins when continuous walking is detected. A prosthesis user starts with a comfortable socket fit at point “A” in the diagram. The socket fit metric (SFM) value at this proper fit is termed the “set point.” The user then gains limb volume during walking and the limb shifts closer to the socket wall, moving to position “B” on the diagram. The auto-adjusting socket reacts by increasing socket size to return the user to the SFM set point, traveling along the blue line to arrive at position “C.” The SFM is now the same as at the start. but the socket is larger because of the person's increase in limb volume. Later, the user sits for an extended period (without socket release), moving to position “D” on the diagram. The person starts walking, and the auto-adjusting socket reacts by decreasing socket size to return the user to the SFM set point, traveling along the green line to arrive at position “E.” The user is now again at the same SFM as at the start, but the socket size is smaller because of the decrease in limb volume during sitting. In experimental embodiments, the auto-adjusting socket sampled at 32 Hz and the maximum adjustment rate was 1 change per second.

To program the auto-adjusting socket for an individual user. characterize the user's plant gain—the change in SFM induced by a change in socket volume (slope of the diagonal lines in FIG. 8). A participant walks at a self-selected speed on a treadmill while the socket is adjusted in 0.25-mm increments across the user's tolerated socket size range. The plant gain is the slope of the least-squares fit to SFM (in counts) plotted against panel position (in mm). Programmed into the socket's microcontroller, the plant gain is used to calculate in real time the change in panel position the auto-adjusting socket should make when the user's SFM deviates from its set point.

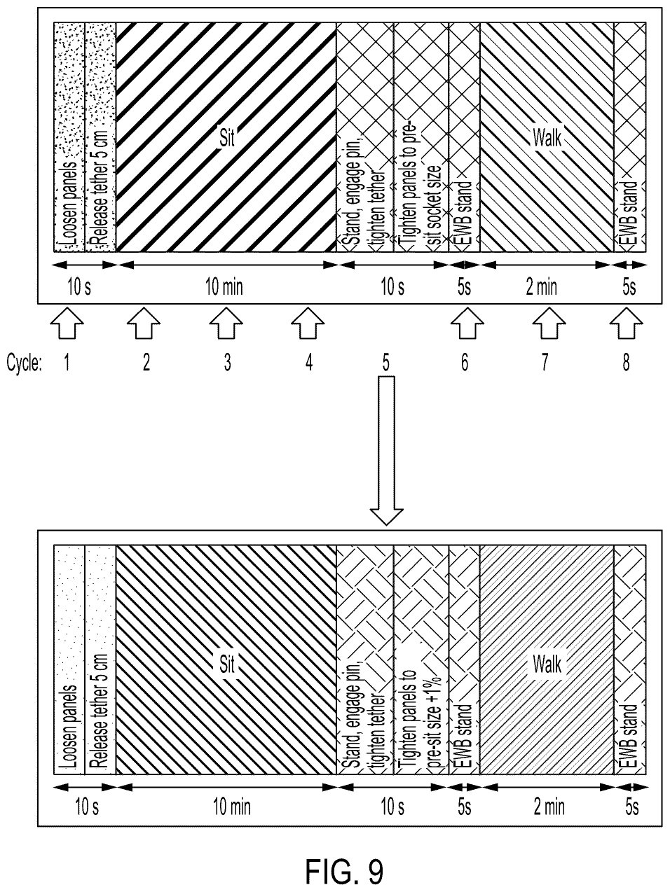

FIG. 9 shows a series of eight sitting and walking cycles in experimental embodiments to observe results of attempts to manage limb volume changes. At the beginning of each sit, the panels were loosened, and the locking pin tether was released 5 cm. The participant then sat for 10 min in a relaxed position with his or her thighs horizontal. knees positioned at roughly the same level as the hips, and feet touching the floor. At the end of the 10-min sit, the researcher drew in the tether using the motor-driven system mounted beneath the socket. The participant stood, then the researcher tightened the panels to the preferred socket size recorded after the plant gain test. The participant stood briefly (5 s) and then walked on the treadmill at a self-selected walking speed for 2 min, activating the control system on the auto-adjusting socket. This sit/walk cycle was repeated, except that at the end of the sit the researcher returned the socket to its size at the end of the prior walk rather than that recorded at the outset of the session. The cycles were repeated until the fifth cycle, where the socket was returned to a size of +1.0% volume larger than that at the end of the prior walk. The appropriate panel adjustment to achieve a +1.0% change was determined using a geometric model of the socket shape. The sixth through eighth cycles were identical to the earlier second through fourth cycles. After the session, participants were returned to their traditional socket and left the lab.

As a subjective measure of the effect of the intervention, participants were asked to provide a relative socket comfort rating (RSCR) at the end of each walking bout in cycles 5-8. The RSCR query was phrased, “Compared to the end of the prior walk, is your socket comfort a lot better, a little better, the same, a little worse, or a lot worse?”

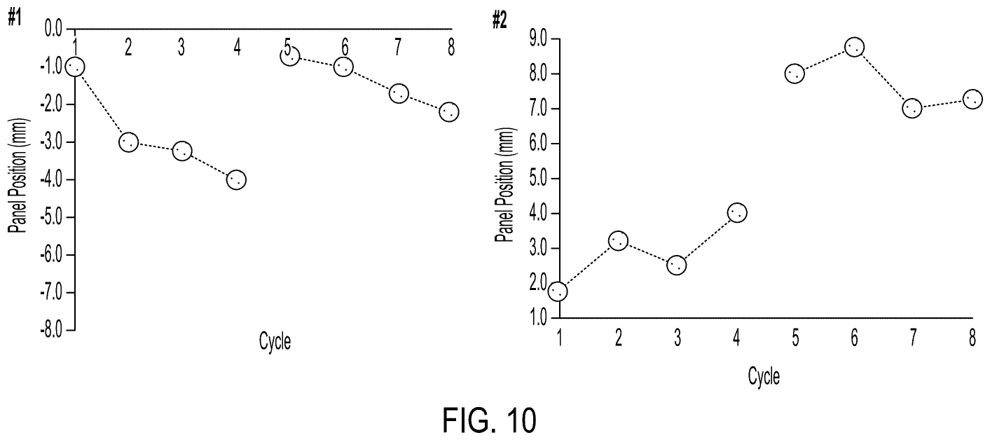

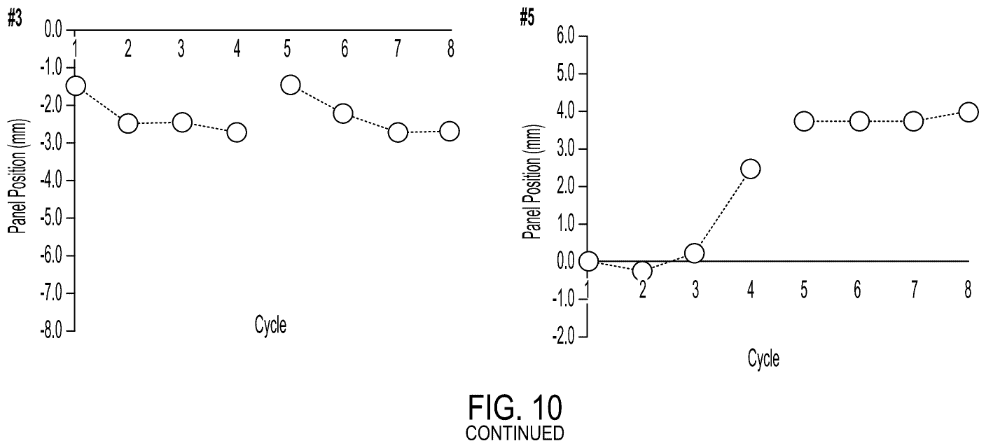

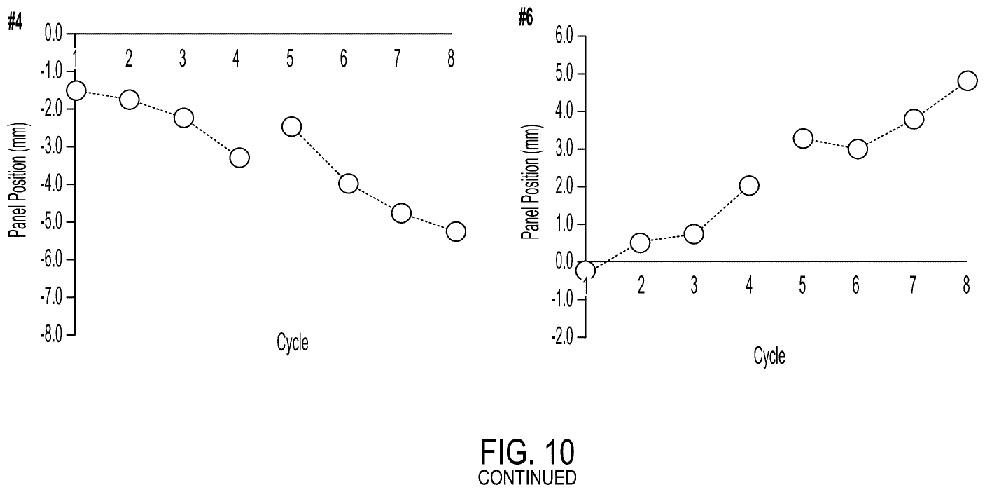

FIG. 10 shows results from 6 participants in a study who worked through the cycles of FIG. 9 and for whom panel position was noted. Panel position increasing indicates the panel loosening the fit of the socket. and for panel position decreasing the socket fit is tightening. Panel position reduced from cycles I to 4 and from cycles 5 to 8 for three participants (#1, #3, #4) and increased from cycles 1 to 4 and from cycles 5 to 8 for three participants (#2, #5, #6) (FIG. 10). Per the specified protocol. the panel position was increased during cycle 5 when the 1.0% relock socket size increase was executed. Panel position data were back onto their trajectory from earlier cycles I to 4 by cycle 6 for participants #4 and #6 and by cycle 7 for participants #2 and #3. Participant #1 did not demonstrate this behavior and instead maintained a larger panel position (larger socket size) compared with cycles 1-4. Panel position for participant #5 stabilized to a consistent distance during cycles 5-8 that was larger than that during cycles 1-4.

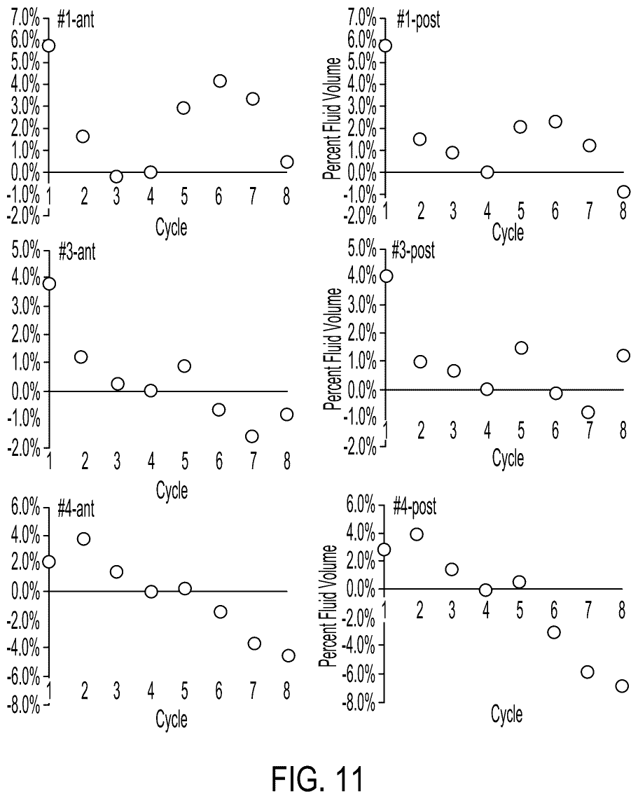

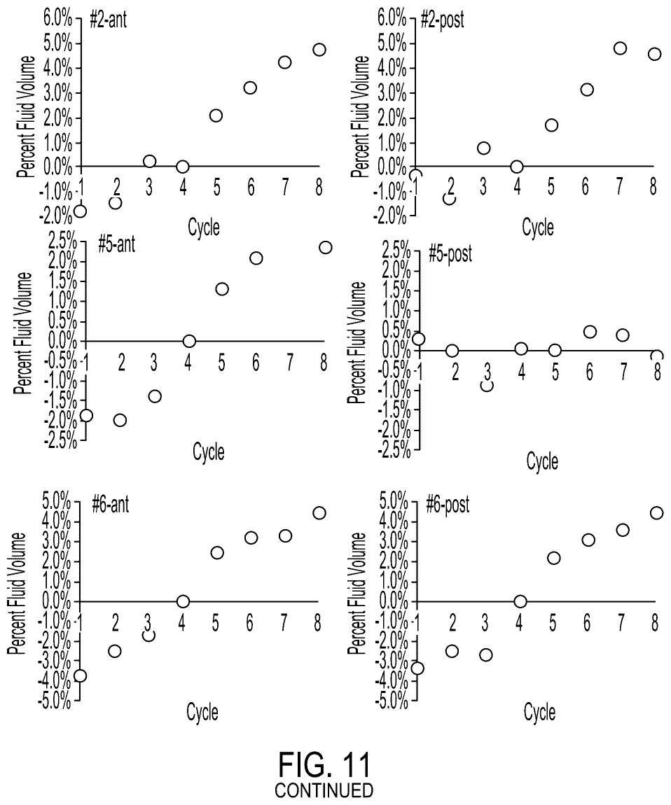

FIG. 11 shows the limb fluid volume for each participant during the same cycles from FIG. 9 as the data from FIG. 10 tracked. Limb fluid volume change over time followed a similar pattern to the panel position data, that is, the patterns of change in FIG. 11 are similar to those in FIG. 10. The shapes of the plots for anterior and posterior regions were similar to each other for each participant except for participant #5 who after the intervention experienced a much greater percent limb fluid volume increase in the anterior region than the posterior region.

SFM was controlled during walking bouts thus it did not follow trends similar to those for panel position or limb fluid volume. As an exploratory effort, we investigated if there was a trend in the change in peak-to-trough SFM (pistoning) from before to after the 1.0% relock socket size increase. For the participants who experienced a loss of limb fluid volume from cycle 1 to cycle 4 (#1, #3, #4), peak-to-trough SFM increased from before to after the 1.0% relock socket size increase (cycle 4 compared to cycle 5). For the participants who experienced a gain of limb fluid volume from cycle 1 to cycle 4 (#2, #5. #6), peak-to-trough SFM decreased from before to after the 1.0% relock socket size increase.

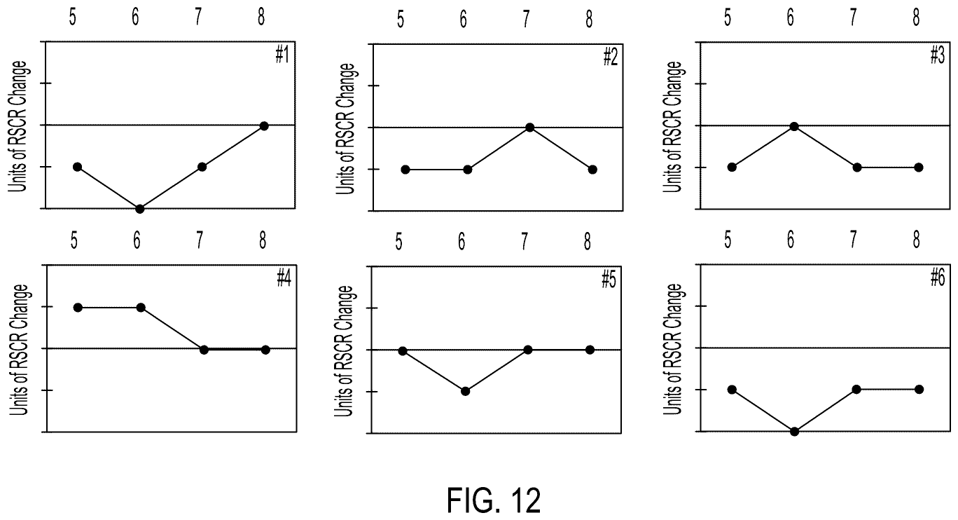

FIG. 12 shows the RSCR for cycles 5 through 8 from FIG. 9, tracked along with the same data as shown in FIGS. 10 and 11. While enlarging the socket by +1.0% after cycle 4 increased limb fluid volume, 4 of 6 participants reported a worsened socket comfort right after it was executed (FIG. 12). Only participant #4 reported an improvement in socket comfort, stating that his socket felt a bit more tightly coupled to his residual limb. Participant #2 reported feeling rubbing on the proximal brim line, participant #3 reported pistoning, and participant #6 reported looseness at the distal end and back part of his limb. Despite the decrease in socket comfort while walking after the 1.0% relock socket size increase, none of the participants decreased their socket comfort score in cycle 8 compared with cycle 5.

As an exploratory effort, we investigated the relationship between percent limb fluid volume change from before to after the 1.0% relock socket size increase (cycle 5-cycle 4) and RSCR. The four participants with percent fluid volume changes greater than 1.0% (#1,2,3,6) all reported reduced RSCR, while the participant with a small increase (#4) (0.6%) reported an increased RSCR. The participant with essentially no percent fluid volume change (#5) (−0.1%) reported no change in RSCR.

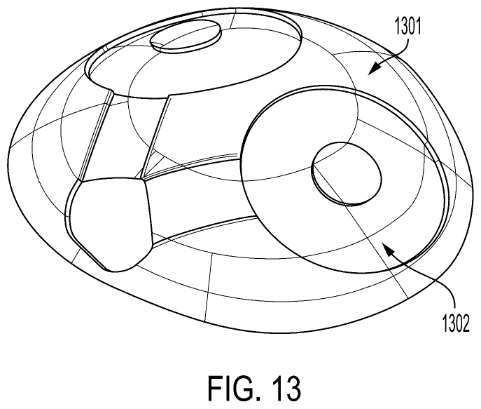

FIG. 13 shows a 3D drawing of an insert 1301 that houses two distance sensors 102 and is placed in the deepest recess of the socket cavity. The insert 1301 is recessed 1302 in the locations where the sensors 102 are placed. The location of these sensors help gauge limb volume and also measure limb motion within the socket. Limb motion is monitored due to the orientation of the two sensors 102 in the insert 1301 being orthogonal to other sensors placed elsewhere in the socket, allowing measuring of movement of the limb up and down in the socket. Contributing to this motion may also be limb volume changes, or weight transfers, but noting any larger changes in movement sensed can contribute to an understanding of the socket fit.

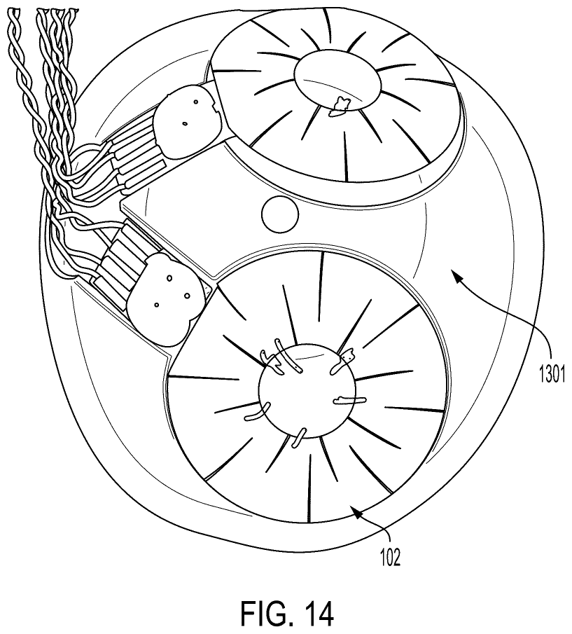

FIG. 14 shows an insert 1301 with sensors 102 in place and ready to install into the prosthetic socket.

### EXAMPLES

### A. Fabricating Sockets with Distance Sensors for Monitoring Prosthesis Use and Socket Fit

The techniques described below have been used to fabricate test sockets for participants in several research investigations. Sensor placement is an intermediate step within the traditional socket fabrication process. It is done after the inner resin layer of the socket (Nyglass ˜0.44 mm thick) is completed and the distal hardware is properly placed. Sensor locations are selected (prefabrication), the mold surface is prepared, antenna and plug locations are marked, the wires and antenna electronics are prepared, the antennae are placed, the sensors are checked to ensure functionality, and the connector plug is placed and potted. A second layup is completed over these elements.

### Prefabrication

During prefabrication, the prosthetist selects appropriate antenna locations. Selection depends on the features of interest to monitor for the specific person. We typically use a “Prosthesis Use” configuration to characterize when and how long people sit, stand, walk, weight-shift, partially doff, and do not use (fully doff) their prosthesis. For the Prosthesis Use configuration, antennae are located at posterior distal medial, posterior distal lateral, anterior proximal medial, and anterior proximal lateral locations (FIG. 3A). We use a “Socket Fit” configuration for signal feedback in an auto-adjusting socket. For the Socket Fit configuration, antennae are located at the anterior patellar-tendon, anterior mid-distal, anterior distal, posterior distal, posterior medial midlimb, and posterior lateral midlimb locations (FIG. 3B). These locations were selected based on results from testing on prosthesis users as described in our previous research.

### Preparation of the Mold Surface

The cured surface is first roughened using a finishing grind cone to sand and remove the smooth coat of resin and expose the inner Nyglass threading. Roughening the surface of resin creates a better bonding medium for antenna and wire placement.

### Marking Antenna and Plug Locations

To properly place the antennae, both the anatomical locations of interest and the physical boundaries of the socket, liner, and suspension system must be considered. If pin-lock suspension is used, a mark is made 2.0 cm proximal from the interface of the four-bolt adapter and the mold on both the anterior and posterior sides. When the pin-locking liner is fully engaged, the liner's umbrella occupies this space. To sense limb-socket distance, the sensors work with a ferrous material in the liner. The ferrous material in the liner serves as the target for the sensor. No portion of the antennae should fall distal of the mark because that would cause the umbrella to overlap the antenna. In that situation, the socket is at a further distance from the ferrous material than at a more proximal part of the liner. An antenna is used to outline placement. If pin suspension is not used, then the four-bolt adapter is attached after the second lamination.

### “Prosthesis Use” Configuration

The Prosthesis Use configuration is intended to record participant bodily positions and activities, including sit, stand, walk, weight-shift, partial doff, and non-use (full doff), as described in our previous work. The Prosthesis Use configuration includes four sensors: two in the posterior distal region equidistant from the midline and two in the proximal region, one medial and one lateral (FIG. 3A). Starting with the two posterior distal placements, the antenna outlines are marked such that their edges are both 5.0 mm from the midline and 0.5 mm proximal to the umbrella-edge mark made previously. In the final socket, the umbrella will likely be further than 0.5 mm distal to the sensor during weight bearing, reducing the risk that the umbrella overlaps the antenna.

Proximal antennae are placed using bony landmarks for reference. The first mark should be placed at the same height as the apex of the medial tibial condyle. The mark's center should be anterior of the direct center of this apex, avoiding high curvature areas as much as possible. The remaining antenna mark (anterior proximal lateral) should be placed such that it mirrors the anterior proximal medial mark and is over the lateral tibial condyle.

### “Socket Fit” Configuration

We have used the Socket Fit configuration in the development of sockets that auto-adjust size to maintain a consistent fit. Six antennae are used: three in the anterior region of the socket and three in the posterior region (FIG. 3B). Outlines for the anterior distal and posterior distal antennae should be placed first. When outlining, one should position the anterior antennae centers along a midline defined by the tibial crest, and the posterior distal sensor along a midline that passes halfway between the hamstrings on the posterior surface. The edges of both the anterior distal and posterior distal antennae should be 0.5 mm proximal from the umbrella edge. If suction or vacuum suspension is used, in which case there is no liner umbrella, there may be space to place antennae at more distal locations. Data from this location may be important if distal limb cyclic motion was of interest. In pilot studies testing suction and elevated vacuum sockets, we have placed sensors at the most distal socket location and demonstrated that the signal provided insight into distal end bearing and vertical motion of the limb in the socket.

The heights of the two posterior midlimb antennae locations are marked. First, a midpoint mark is made along the posterior midline midway between the lowest trimline on the posterior socket brim and the interface of the four-bolt adapter with the foam model. The two posterior midlimb antennae are outlined with centers equidistant from the posterior socket midline, medially and laterally, and their centers at the height of the midpoint mark. The two posterior sensors should be outside the hamstrings. Another mark is made on the anterior midline, at the same level as the posterior midpoint mark. The anterior midlimb antenna should have its center at the anterior midpoint mark. The remaining anterior antenna, the anterior patellar-tendon antenna, should fall in alignment with the anterior midlimb and anterior distal antenna and be at a level between the patellar-tendon-bar and the tibial tuberosity.

Lastly, for both the Prosthesis Use and Socket Fit configurations the location for the socket plug is marked. The socket plug should be on the lateral aspect of the mold because the electronics boards will be connected on this side. Medial placement may be bothersome for some users. The mark should be placed about 2.0 cm proximal from the interface of the four-bolt adapter with the foam positive.

### Preparation of the Wires and Antenna Electronics

The routing path for the antennae's lead wires must be established. The twisted-pair wires are routed along the socket surface from each marked antenna location to the socket plug mark, making sure wire routes do not overlap. Chosen wire routes should minimize distance between the sensor and the plug. An additional 2.0 cm is added to each measured wire-route length to ensure that the wires are not taut when they are adhered to the mold.

Once the wires are cut, they must be soldered to the antennae. Wires are first stripped ¾ the length of the antenna's solder pad, about 5.0 mm. The twisted-pair wire is unraveled slightly to align the wires along the solder pads. A capacitor and thermistor are soldered onto the antenna, completing the wiring process.

### Antenna Placement

Working one at a time, antennae are adhered to their marked locations using double-sided tape. Antennae in high curvature locations, such as the distal end of the socket, may require a center cutout to allow the antenna to be curved to fit the shape.

The solder pads of the antennae are adhered to the mold using double-sided tape. The solder pad connections are potted with hot melt glue. It is important to flatten the hot melt while still pliable so that it conforms to the shape of the surface, minimizing any bumps or sharp edges once hardened. The hot melt should be shaped to a thickness of about 1.5 mm. At about 2.0 cm of wire length away from the solder tabs to the antennae, the connections are strain-relieved by adhering the wires to the mold using hot melt glue. Strain-relieving the wires minimizes the risk of mechanical failure of the electrical connections. Remaining wire length extending to the connector is routed and adhered with hotmelt, taking care to avoid overlapping wires and inducing excessive tension. Once all wires have been routed and adhered toward the plug (each wire should still have an extra 2.0 cm of length at this point), each wire is oriented in its connection order in the plug The connections in the plug are ordered by sensor/thermistor pair. The connections for one sensor are made sequentially before making the connections for the next sensor. This step is done so that the remaining wires do not become crossed once cut and crimped. The antenna wires, along with an additional ground wire that is woven through the carbon fiber weaves taking care not to split tows, are then cut and crimped. The ground wire is kept away from the antennae.

### Sensor Check to Ensure Functionality

Antenna wires are connected to the plug, starting with the ground wire. The plug is connected to the electronic circuitry and tested by positioning a ferrous target near the antennae. The electronic circuitry includes an inductive sensing chip (LDC1614, Texas Instruments, Dallas, TX, USA). associated electronics, a battery, and a data storage medium. We use a custom data logger described in our previous work. If an antenna is not functional, typical reasons are a damaged wire near the crimp and a failed solder point such as at the wire-antenna connections or at the capacitor and thermistor connections to the antenna.

Once all the sensors are deemed functional, ferrite covers are placed over the antennae. A small gap is left so that the ferrite pads do not contact the exposed electronics (capacitor and thermistor) or via between them and the antenna. Hot melt glue is used to pot the exposed capacitor and thermistor and via sealing the gap.

### Placing and Potting the Plug

Before the second lamination, the plug must be adhered to the mold's surface using hot melt glue. The plug should be protected, for example, within a custom 3D-printed housing. The mold is oriented so that the socket axis is parallel with the ground and the connector plug mark made previously is pointing upward. Hot melt is then applied onto the mold surface. Sufficient hot melt is needed to ensure the plug does not move during the second lamination. The plug is placed parallel to the bottom of the foot on the posterior lateral aspect of the socket, taking care to minimize strain on the wires, and adhered. The plug is potted using a platinum cure silicone (Dragon Skin 10 FAST, Smooth-On, Macungie, PA, USA) to prevent resin from entering the plug. If additional hardware is to be added (e.g., a ratcheting dial for a cabled-panel socket) then the placement of the plug may need to be adjusted accordingly.

### Preparation for the Second Lamination

The second lamination seals the exposed sensing system elements, protecting them from mechanical damage. First, a temporary plastic cover is placed over the four-bolt adapter. The temporary cover is used to keep the four-bolt adapter clean until the last layup, where it is removed so that the layup conforms around the adapter for structure. A piece of Nyglass is wrapped around the base of the plug, where it connects to the mold. The Nyglass adds structural support, which will be needed during the second lamination process. A single layer of woven carbon fiber is then tied-off proximal to the four-bolt adapter and reflected down over the socket mold—making sure to remove any bunching, which could lead to sharp edges once laminated. The ground wire attached to the plug during previous steps is threaded into the carbon fiber tows, which are spread around the plug. Spreading the tows around the four-bolt adapter is a common socket fabrication technique for the final layup.

Putty and tape are used to seal the plastic covered four-bolt adapter to keep resin out during the second lamination. The polyvinyl acetate outer bag is draped over the mold with care to avoid introducing excess carbon fiber around the four-hole adapter. Bunching of the bag or carbon fiber around the connector should be avoided. The bottom of the bag (proximal end of the socket) is sealed off using tape and a vacuum is pulled. Resin is poured, and the top of the bag is sealed while the resin sets. If additional hardware needs to be added, like a ratcheting dial for a cabled-panel socket, then it is placed on the cured carbon layer over the sensors. After placing this hardware, the technician should cover the hardware with an additional layup made up of one carbon fiber layer, two Nyglass layers, and one carbon fiber layer. If no additional hardware needs to be added, then the last layup covering the carbon fiber should be two Nyglass layers and one carbon fiber layer.

### Pairing the Instrumented Socket with a Ferrous Liner

Once the last lamination is completed, the socket is ready to be paired with the ferrous liner with which it will be worn. We use two simple calibration tools to pair the liner with the socket, similar to those described in our previous work. Briefly, a bench test setup designed to move an antenna fixed distances from the liner is used to establish a calibration curve. Then the liner is placed over an inflatable balloon in the shape of a residual limb and the assembly is put in the socket. Data are collected while the balloon is brought to a pressure sufficiently high enough to push the liner against the socket but not so high that the liner is pushed proximally out of the socket. Data collected under this condition establish the offset (y-intercept) of the calibration curve. The calibration curve is used to process and convert participant data into sensed distance in units of mm. Once the calibration procedure is completed, the socket is ready for regular use.

### Conclusion

The described methods for placing inductive distance sensors within prosthetic sockets for people with transtibial amputation are intended to facilitate further research use of this technology. Embedded socket sensors may provide a long-term monitoring option for practitioners to obtain knowledge of their patients' historical or present status in their free-living environment and community. As described by Hafner and Sanders, this insight may augment the patient-reported experience with reliable data that are unencumbered with the limitations of patient recall. Ultimately, this sensing system may better inform individualized treatment strategies and help establish objective rationale for prescription of specific prosthetic components intended to optimize a patient's function, health, safety, and quality of life.

### B. A Novel Portable Sensor to Monitor Bodily Positions and Activities in Transtibial Prosthesis Users Instrumentation

Custom-designed distance sensing elements, termed sensors, were adhered to the inside of the socket. Participants wore a ferrous elastomeric liner, a liner with a trace amount of iron powder embedded in the outer layer. The iron powder served as a distance target for the sensing elements. A custom data logger, enclosed within a carbon-fiber cover, was fastened to the anterior lateral aspect of the socket. Data collected and stored to the unit were later downloaded and processed to identify bodily positions and activities.

### Sensor

A sensor is a custom flexible coil antenna of diameter 32.0 mm and thickness 0.15 mm that we designed in prior work. A surface mount capacitor (220 pf) and a thermistor (10 kΩ) are soldered to the sensor near the antenna coil. The antenna, which is an inductor (L), and the surface mount capacitor (C) connect through a flexible conductor to the data logger. The intensity of the signal is related to the distance between the antenna and the iron powder target embedded in the liner. Thermistor data is stored as a separate channel in the data logger.

### Ferrous Liner

Participants wore a pin-locking silicone elastomeric liner (Alpha series, WillowWood, Mt. Sterling, Ohio, USA) with trace amounts of iron powder embedded within a thin outer portion of the elastomer. The ferrous liners purchased for this study functioned similar to liners that we developed in our prior work and used in an auto-adjusting prosthetic socket.

### Echo

The ECHO is a data logger that provided battery power to the unit, sampled the sensors, and stored collected data. An inductive sensing chip drove the antenna and capacitor, creating a LC tank oscillator. The presence of the magnetically permeable target within the sensor's field reinforced the inductor and lowered the oscillation frequency in a distance-dependent manner. The change in frequency measured by the inductive sensing chip was a measure of distance between the sensor and iron powder target. The system sampled at a rate of 32 Hz and stored collected data to a micro-SD card.

### Protocol

Participants came to the lab and conducted a scripted protocol and a semi-scripted protocol. Then they started take-home testing. The research practitioner first inspected the participants' residual limb and gait to ensure inclusion criteria were met. Participants sat while wearing their prosthesis for 10 min while the research practitioner collected basic demographic data and information about any recent changes to the prosthesis. Participants were asked to estimate how long they wore their prosthesis each day. Participants then doffed their prosthesis but not their liner.

The prosthesis was instrumented with four sensors adhered to the inside of the socket using double-sided adhesive tape (SpeedTape, FastCap, Ferndale, Washington, USA). The posterior distal lateral and posterior distal medial sensors were placed on the posterior wall of the socket, just above the edge of where the liner umbrella would contact the socket. The edge of each was 5.0 mm away from the midline. The anterior lateral proximal sensor was placed at the tibial plateau lateral of the tibial tuberosity, and the anterior medial proximal sensor was placed equidistant medial of the tibial tuberosity. A protective, self-adhesive, low friction material (ShearBan, Tamarack, Blaine, Minnesota, USA) was placed over each sensor.

After the sensors were placed and functionality confirmed, participants were asked to execute a scripted protocol sitting and standing in different bodily positions. A video camera (HERO 3, GoPro, San Mateo, California, USA) was run continuously so that bodily positions could be confirmed later during data analysis. Four seated positions were tested: relaxed; foot in front of knee; foot even with knee (90°); and foot behind knee. Participants sat in these four positions in three different chairs in the following order: a chair fitted to participants' height; a short chair; and a tall lab stool. For the short chair the knee was flexed less than 90°, and for the tall lab stool the knee was extended beyond 90°. Participants stood in three weight-bearing conditions on their prosthesis: low weight-bearing, equal weight-bearing, and full weight-bearing.

Participants next conducted a semi-scripted protocol for approximately 40 min. They were asked to walk with the researcher to three different locations outside of the lab and to rest for 8 min at each location. The walks included 1-min elevator rides and a variety of walking surfaces (linoleum, cement, gravel, and dirt) with stairs and different inclines that met American Disabilities Act (ADA) wheelchair ramp standards. The paths were intended to reflect a range of walking conditions prosthesis users may experience in their everyday lives. The three locations were intended to provide different environments that may have stimulated participants to stand and sit in different bodily positions. Participants were not given specific instructions about how or where to walk and sit but instead did what was comfortable to them. The researcher wore the video camera in a chest-mounted harness that recorded the participant throughout the protocol.

Participants left the lab and continued to wear the instrumented prosthesis in their at-home environment for up to 7 d. They were instructed to charge the system nightly. After the test period, the participant returned to the lab, the sensors and electronic enclosure were removed, and the participant left the lab.

### In-Lab Scripted Protocol

Results from the in-lab scripted protocol included data for walking and a variety of bodily positions. Time points in the data stream at the beginning and end of each 5 s of a bodily position were identified. A mean value was calculated for each sensor, and the results were plotted over time. The video data were inspected carefully, and for each bodily position, descriptions were written of the orientation of the participants' torso, position of their feet, and angle of their knee. These descriptions were used later to identify bodily position from data collected during the 40-min semi-scripted protocol.

### Out-of-Lab Semi-Scripted Protocol and Take-Home Testing

For both the 40-min out-of-lab semi-scripted protocol conducted and the take-home testing, data were segmented into sections of sits, seated shifts, stands, standing weight-shifts, walks, partial doffs, and non-use (full doffs). A seated shift is movement while sitting. A standing weight-shift is movement while standing. In the algorithm, select magnitudes were used to determine two of the variables—partial doffs and non-use. Peak and trough thresholds that adjusted over time were used to detect walking bouts, sits and stands. A confusion matrix, a technique for assessing the performance of a classification algorithm, was used to optimize selection of the sit-stand threshold (FIG. 5). We compared confusion matrix results with results using a sit-stand threshold determined by visual inspection of the data. Sit shifts and standing weight-shifts were identified using the derivative of the sensed distance data. The algorithm is described in detail elsewhere in this detailed description.

For each participant, the time spent daily in each activity and bodily position was calculated and plotted in a bar graph. Total time for each activity and bodily position was also plotted as a percentage of the sum of all prosthesis day durations. As described in the results below, data from the semi-scripted protocol demonstrated that stands immediately after doffs were poorly classified. Thus, for the take-home data, if a stand was identified after a doff but before a walking bout of at least 5 steps, then the data in between was classified as a seated shift or standing weight-shift using the criteria described in the detailed description (FIG. 5). Steps were identified during both walking bouts and during standing weight-shifts within the developed algorithm. They were tabulated as bout steps or standing weight-shift steps.

### Results

Four individuals (one female and three males) participated in this study. All participants had their amputation as a result of trauma, and all used locking pin suspension. Median limb circumference and residual limb length were 26.9 cm and 15.2 cm, respectively. Median age was 42.5 y, median time since amputation was 14.0 y, median participant weight was 71.5 kg, and median height was 173.0 cm. Because participant #2 was pregnant, she did not conduct the out-of-lab semi-scripted protocol. Instead, she conducted a shortened version of that protocol in a 20-min session in the lab. Because she found it uncomfortable to stand still, she conducted very few stands. Participant #1 was not able to execute the sit relaxed position in the short chair. He conducted an additional cycle of sitting positions in the tall lab stool.

From visual inspection of the data, we observed that the proximal sensor pair more effectively distinguished sitting from standing than the distal pair for three of the four participants. For one participant (participant 1), the participant with the least distal soft tissue, the distal sensor pair more effectively distinguished sitting from standing. For all participants, the distal sensor pair more effectively distinguished standing weight-bearing levels from each other (low, equal, full).

The system did not well differentiate the various sitting positions from each other. Particularly for the proximal sensors, standing data for equal and full weight-bearing were clustered near the stance phase minima during walking. In general, low-weight-bearing standing showed a higher sensed distance than full or equal weight-bearing.

The sit-stand threshold identified using two methods, visual inspection of the data and the confusion matrix optimization (FIG. 5), expressed as a percentage of the range of the envelope (maximum minus minimum). All differences are within 7%.

Using the proximal sensor pair for participants 1, 2, and 4 and the distal sensor pair for participant 3 in the confusion matrix optimization, sit detection accuracy was 95.7% for participant 3 and 100.0% for participants 1, 2, and 4. At least 90.0% of the stands were detected correctly for participants 1, 3, and 4, but only 62.5% for participant 2, the participant who executed very few stands. Including stands detected within 5 s after dons diminished classification accuracy for all participants.

For step detection, missed steps occurred primarily during walking bouts of five or less steps. The few actions that were erroneously identified as steps were typically detected instead as seated shifts. For all participants, at least 95.6% of the steps were accurately detected.

The system monitored activity for between 4 and 7 days for all participants. Data collection was stopped when the system no longer collected data. This occurred when the sensor leads mechanically failed, which was typically where they curved over the socket brim. One of participant 4's anterior proximal sensors was mechanically unstable, so the distal sensor pair was used for activity detection. Detection accuracies for the distal pair were comparable to those for the proximal pair, except for sit detection which was 91.7% instead of 100%.

Participant estimates of daily prosthesis wear were 18, 12, 16, and 12 h for participants 1 to 4, respectively. Participant estimates of prosthesis daily wear were longer than the prosthesis wear duration calculated from the data for 3 of the 4 participants, with difference ranging from 1.7 to 4.3 h. Participant 2 underestimated prosthesis wear by an average of 2.6 h.

The percentage time for the different bodily positions and activities varied between participants. Interestingly, the two participants with the highest percentage time doffed (participants 3 and 4) also had the highest percentage time walking, standing weight-shifting, and standing.

Bout step counts were strongly related to walk duration, indicating that participants maintained a consistent cadence across days. The mean cadence across days was 41.8 steps/min for participant 1; 45.9 steps/min for participant 2; 43.9 steps/min for participant 3; and 42.2 steps/min for participant 4. The normalized root mean square error (to the mean) (NRMSE) between walking time (min) and bout steps (count) was low, 0.04.

The percentage of all steps that occurred during standing weight-shifts, as opposed to during walking bouts, was 28%, 15%, 33% and 34% for participants 1 to 4, respectively. Step count did not well reflect the time participants spent actively using their prosthesis, i.e., the sum of time walking, standing, and standing weight-shifting (FIG. 6b). Total steps were not strongly related to active use time. The NMRSE between active use time (min) and total steps (count) was high, 1.00.

### Discussion

The purpose of this study was to develop and test a system capable of building upon traditional measures of physical activity (i.e., step count and cadence) in order to better characterize how a person uses their prosthesis. Our work extends from previous efforts using a commercial load cell on participants with osseointegrated prostheses to try to characterize prosthesis use. Previous works delineated weight-bearing motion within a limited space (localized locomotion) from walking (directional locomotion) and stationary loading.

On average, patient's estimates of daily prosthesis use differed by 5.8 h from that measured by the monitors, higher than the difference between patient estimates of daily prosthesis wear and monitor data in the present study, 3.0 h. The more specific question in the present study, querying the participant about “wear” rather than “use” may explain the improved results. Rigorous scientific investigation would be needed to confirm the interpretation that this difference in wording affects the results.

Because the sensors measured liner-to-socket distance even when there was little or no contact pressure, we easily distinguished times when the prosthesis was donned, partially doffed, and entirely doffed (i.e., not used). These distinctions are relevant because the different donned states deliver different stresses to residual limb soft tissues, affecting both tissue health and limb volume. Interestingly, the two participants with the greatest step count and active use time in the present study (participants 3 and 4) also had the greatest non-use time, suggesting this relationship should be investigated in larger studies.

We expect that a main reason the proximal sensors performed better than the distal sensors on most of the participants in this study to distinguish sitting from standing is because the proximal sensors were located over the tibial plateau, a location with little soft tissue over the bone. A change in residual limb fluid volume is unlikely to affect tissue thickness at this location. At distal locations, however, there is in general more soft tissue and thus sensed distance is likely more affected by limb fluid volume changes. This sensitivity makes it difficult to distinguish if a decrease in distal sensed distance occurred because residual limb volume decreased and the tibia displaced distally in the socket, or if distal limb fluid volume increased and tightened the distal socket fit. This uncertainty is likely more relevant for people with moderate to high soft tissue at the distal end of the residual limb. This interpretation is supported by the data from the present study. Participant 1, the participant with the least distal soft tissue, was the only participant for whom the distal sensor pair better distinguished sits from stands. The other three participants had moderate to high distal soft tissue.

The sensor's strong sensitivity to changes in distance (LDC counts/mm) allowed us to distinguish walking bouts, standing weight-shifts, and stands, three very different uses of a prosthesis. While participant percent time walking was comparable among the four participants, ranging from 4% to 7% of their prosthesis day, their standing weight-shift and stand times varied, highlighting the difference in prosthesis use among these four individuals. This in part explains why step count data did not well-reflect active prosthesis use. We also note that the percentage of steps during walking bouts, as opposed to during standing weight-shifts, was considerably higher for one participant (participant 2) (85%) than the others (73%, 57%, and 65% for participants 1, 3 and 4, respectively). Participant 2's energy expenditure while taking steps may have been higher than the others even though she took fewer total steps, though rigorous scientific investigation would be needed to demonstrate that walking is more energy consuming than weight-shifting. This more detailed information of how a person uses their prosthesis, not just how many steps they take, may be of clinical utility for a range of evaluative, diagnostic, and prognostic applications.

Distinguishing walking bouts from standing weight-shifts may also facilitate programming of auto-adjusting prosthetic sockets. Unlike walking, standing weight-shifts would not be expected to stimulate a residual limb volume increase because they are not sufficiently strenuous to the cardiovascular system. They do not increase vascular drive as much as walking, which has been shown to facilitate fluid volume recovery particularly in people without vascular co-morbidities. We postulate that for prosthesis users who tend to lose limb volume over the day, increasing socket size during standing weight-shifts will not improve socket fit whereas increasing socket size during walking bouts will help stabilize fit. This is conjecture, however, and rigorous scientific investigation is needed to determine best practices for programming auto-adjusting sockets.

While a person sits with their prosthesis donned, the prosthesis is used to stabilize both legs and to change position, for example to execute a task or to relieve pain in the joints. It is important for the person to have both legs supporting the body while seated to reach items, bend down to tie a shoe, or move a wheeled desk chair around a workspace, for example. Residual limb soft tissues may be put at risk if interface stresses are concentrated when a prosthesis user sits with the socket donned. If the foot is positioned behind the knee and the knee flexed, for example, the patient may be at risk of pressure ulceration over the distal tibia. Placing the foot flat on the floor may improve stability in transition to standing. Though the sensors did not well identify different sitting positions in this study, they did well delineate sitting from standing. We would expect that simple modifications such as adding an inclinometer to monitor lower leg orientation would improve success identifying different sitting positions during detected sits. Distal sensor data may help determine if the person is leaning into the socket during sitting, possibly increasing breakdown risk.

A limitation of this study was that two of the four participants did not execute the complete in-lab scripted protocol, changing the amount of data available for one of the sitting positions for participant 1 and the amount of standing data available for participant 2. The missing data for participant 1 was not expected to affect the interpretation of the data, or the conclusion that an inclinometer should be added to the system to help lower-leg orientation during sitting. The lack of sufficient standing data available for participant 2 may help explain her weaker stand detection performance compared with the other participants.

Other sensing modalities besides inductive distance sensing have been pursued to try to distinguish sitting and standing. Efforts include measuring from two inclinometers, one on the calf and one on the thigh, adding inertial measurement units and gyros (e.g., GT9X, Actigraph), using in-shoe pressure sensing mats, and placing load cells in or above the pylon. However, these modalities do not differentiate a donned from a doffed or partially doffed prosthesis. Monitoring the limb-socket interface, as in the present study using the sensors, accomplishes this objective.

Next steps extending from this initial study are to enhance the durability of the sensing system to enable long-term at-home monitoring. A means to protect the sensor leads where they reflect over the socket brim would make the system more durable. Embedding the sensors during socket fabrication is another alternative to enhance durability and allow for long-term monitoring.

### Conclusion

The inductive sensing system measures bodily position and type of activity during at-home clinical monitoring, including sit, seated shift, stand, standing weight-shift, walk, partial doff, and non-use. Data collected from participants suggests that step count and cadence data may provide an incomplete picture of prosthesis use, and that the bodily position and activity data may help fill the void.

### C Performance of an Auto-Adjusting Prosthetic Socket During Walking with Intermittent Socket Release

### Socket Fabrication

Each participant's traditional socket was scanned so that we could duplicate its shape for the investigational prosthesis. The investigational prosthesis was fabricated with three adjustable panels located on load-tolerant areas of the residual limb (anterior medial, anterior lateral, and posterior midline) (FIG. 3A). Panel size was maximized so as to impact socket volume change while avoiding bony prominences at the anterior distal tibia, fibular head, and tibial crest—areas that may be sensitive to compression. Sensors that measured the distance between the liner and socket termed socket fit sensors, were positioned within the socket wall during fabrication at the posterior medial mid-limb, the posterior lateral mid-limb, and the anterior distal limb. The stance phase minima from the two posterior channels were used in the automatic, panel position adjustment algorithm. The anterior distal channel was used to detect walking, implemented the same way as in our previous study. All sockets were made with tether suspension.

To adjust the socket size, we placed direct current (DC) micromotors in frames that spanned over each panel. Each frame was affixed to the outside of the socket using custom threaded mounts positioned within the socket wall during fabrication. Each motor included an encoder and gearhead and weighed 26 g (model 1717006SR 1EH2-4096 15A152:1+MG03, Faulhaber (Micromo), Clearwater, Florida, USA). The motor unit was of diameter 17.1 mm and length 40.8 mm. The frames and motors added 865 g to the overall weight on the socket. The motor drove gearing and a winch assembly that translated the motor's rotation into radial displacement of the panel, as described in our prior work. Unlike cabled-panel sockets, this design allowed the panel to be pulled radially outward beyond the surrounding socket and relieve panel contact with the residual limb. Further, a universal joint at the connection of the panel to the winch minimized stress concentrations at the edge of the panel. Because of these design features, no cushioning material needed to be placed on the inside surface of each panel as with a traditional cabled-panel socket. A cable connection to a PC ran a virtual instrument (VI) (LabVIEW National Instruments) that adjusted the panels in 1-step increments. 28 Each step induced a 0.25-mm radial displacement in each of the three panels. A panel position of 0.00 mm was defined as the position where the panels were flush with the surrounding socket. When the socket was put in auto mode, which was activated using the VI, the control scheme described below was implemented.

### Auto-Adjustment Algorithm

The auto-adjustment algorithm operated during all walking bouts. It started when continuous walking was detected. It operated using the VI, implementing a custom program similar to that described in our previous work. The diagram in FIG. 8 illustrates how the auto-adjusting socket operated. Consider a user who starts with a comfortable socket fit at point “A” in the diagram. The socket fit metric (SFM) value at this proper fit is termed the “set point.” The user then gains limb volume during walking and the limb shifts closer to the socket wall, moving to position “B” on the diagram. The auto-adjusting socket reacts by increasing socket size to return the user to the SFM set point, traveling along the blue line to arrive at position “C.” The SFM is now the same as at the start, but the socket is larger because of the person's increase in limb volume. Later, the user sits for an extended period (without socket release), moving to position “D” on the diagram. The person starts walking, and the auto-adjusting socket reacts by decreasing socket size to return the user to the SFM set point, traveling along the green line to arrive at position “E.” The user is now again at the same SFM as at the start, but the socket size is smaller because of the decrease in limb volume during sitting. The auto-adjusting socket sampled at 32 Hz. The maximum adjustment rate was 1 change per second.

To program the auto-adjusting socket for an individual user, we first executed a test in the lab to characterize the user's plant gain, the change in SFM induced by a change in socket volume (slope of the diagonal lines in FIG. 8). The participant walked at a self-selected speed on a treadmill while the researcher adjusted the socket in 0.25-mm increments across the user's tolerated socket size range. The plant gain is the slope of the least-squares fit to SFM (in counts) plotted against panel position (in mm). Programmed into the socket's microcontroller, the plant gain is used to calculate in real time the change in panel position the auto-adjusting socket should make when the user's SFM deviates from its set point.

As described in the testing protocol below, the researcher operated the VI via a computer interface to adjust socket size during sitting between bouts of walking. In addition, a motor-driven system mounted beneath the socket, similar to that described in our prior publication, was used to draw in and release a tether to the liner.

### Testing Protocol

Once the investigational prosthesis was fabricated and instrumented, the participant visited the lab for a fitting and evaluation session. The research prosthetist evaluated the participant's gait and adjusted alignment of the foot and length of the prosthesis if needed. The participant walked on the treadmill at different panel positions (socket sizes) to ensure the socket was comfortable and to ensure the instrumentation performed properly.

On a separate day, the testing protocol was conducted. After arriving at the lab, participants sat for at least 10 min with their traditional prosthesis donned to achieve a homeostatic condition. Participants then doffed their prosthesis, and their residual limb was instrumented with thin surface electrodes that are part of a bioimpedance system which was used to monitor limb fluid volume as described in detail in prior publications. The electrodes were configured to monitor the anterior region and the posterior region of the residual limb. Data collection from the limb fluid volume monitoring system and the socket fit sensors was initiated. A plant gain test was conducted, then the socket was returned to the participant-preferred socket size. The researcher then used the VI to put the socket in the auto-adjustment mode.

A series of eight sitting and walking cycles was conducted (FIG. 9). At the beginning of each sit, the panels were loosened, and the locking pin tether was released 5 cm. The participant then sat for 10 min in a relaxed position with his or her thighs horizontal, knees positioned at roughly the same level as the hips, and feet touching the floor. At the end of the 10-min sit, the researcher drew in the tether using the motor-driven system mounted beneath the socket. The participant stood, then the researcher tightened the panels to the preferred socket size recorded after the plant gain test. The participant stood briefly (5 s) and then walked on the treadmill at a self-selected walking speed for 2 min, activating the control system on the auto-adjusting socket. This sit/walk cycle was repeated, except that at the end of the sit the researcher returned the socket to its size at the end of the prior walk rather than that recorded at the outset of the session. The cycles were repeated until the fifth cycle, where the socket was returned to a size of +1.0% volume larger than that at the end of the prior walk. The appropriate panel adjustment to achieve a +1.0% change was determined using a geometric model of the socket shape. The sixth through eighth cycles were identical to the earlier second through fourth cycles. After the session, participants were returned to their traditional socket and left the lab.

As a subjective measure of the effect of the intervention, participants were asked to provide a relative socket comfort rating (RSCR) at the end of each walking bout in cycles 5-8. RSCR has been used previously in prosthetics research to study relative changes in socket comfort within a session. The RSCR query was phrased. “Compared to the end of the prior walk, is your socket comfort a lot better, a little better, the same, a little worse, or a lot worse?”

### Data Processing and Analysis

Data collected across the test session, including the SFM, set point, and panel position, were downloaded from the auto-adjusting socket and plotted over time for visual inspection. The walking portion of each cycle was extracted for further analysis. For each session, the range of panel position was calculated. For each bout, the absolute error of the auto-adjusting socket control system (SFM minus set point) was plotted over time and the IAE calculated as

$$\overline{IAE} = \frac{1}{N} {\sum }_{i = 0}^{N} \vert {SFM}_{i} - {SFM}_{0} \vert$$

where SFMᵢ is the measured SFM of the i^{th} temporal index, SFM₀ is the SFM set point, and N is the number of data points in the analysis. Thus, N for an IAE calculated during the first 30 s of a bout included all data points up to 30 s, while N for an IAE calculated during a whole bout included all points in the bout.

Limb fluid volume data from the bioimpedance system were downloaded and converted to extracellular fluid volume using de Lorenzo's form of the Cole model. The data were time-synchronized with the SFM data. The minimum fluid volume during stance phase of each step was determined for both the anterior and posterior limb regions and a mean calculated for each bout. The means for a session were expressed as a percentage change relative to the mean fluid volume during cycle 4, the cycle before the 1.0% relock socket size increase. This strategy allowed a consistent reference across participants for the percent fluid volume change between cycle 4 and subsequent cycles, a variable of interest in this study.

Relative socket comfort rating data were expressed as a change relative to cycle 4, the cycle before the relock socket size increase was executed. “A little better” and “a little worse” were defined as a plus one-unit change and a minus one-unit change, respectively. No participants responded with “a lot better” or “a lot worse,” so no unit change was defined for them.

### Results—Participant Panel Positions, Limb Fluid Volume, and Pistoning

For 5 of the 6 participants, the range of panel position (maximum-minimum) during walking across the test session was between 2.26 mm and 6.01 mm. For the remaining participant (participant #2), the control system saturated at the maximum panel radial distance (10.00 mm) for part of one walking bout, contributing to a higher range, 8.75 mm. Panel position range was not well-correlated with plant gain (R=0.38). Results are discussed above in paragraphs [0060]-[0066] with respect to FIGS. 9-12.

### Discussion

In this study, conducting socket release during sitting between bouts of walking was shown to have the intended effect of changing participants' limb fluid volume. In general, the auto-adjusting socket responded well to these perturbations and maintained stable performance and low error. We believe the results warrant advancing to testing on prosthesis user participants in their at-home environments, increasing the number of participants and determining the long-term clinical outcomes in terms of comfort, prosthesis use, skin health, and other related outcomes.

Part of the reason the control system demonstrated stable performance and low error was because fluid volume change followed panel position change as shown by the similarity of curve shapes in FIGS. 10-11. Our prior work and the plant gain tests in the present study showed that, when socket size adjustments were made. the distance sensed by our custom sensors changed linearly with socket size. Taken together, these results suggest that the control system operated as intended for all participants—adjusting panel position based on information from the distance sensors served to adjust limb fluid volume.

It is recognized that clinically it may not be desirable to increase limb fluid volume over time, as occurred for participants #2, #5, and #6 (FIG. 11). However, what is relevant to note (and was the purpose of the present study) is that the auto-adjusting socket is capable of being used to control limb fluid volume. The auto-adjusting socket worked in harmony with the residual limb to take advantage of the limb's capability for fluid volume change to accomplish this result. In clinical practice, the auto-adjustment algorithm would be programmed to maintain a consistent sensed distance over the day.

We expect that the use of distance sensing and the practice of not placing the sensing elements on the actuators are two key reasons the auto-adjusting socket in this study performed better than other systems described in the literature. Distance sensing may be more effective than pressure sensing because the measurement is very sensitive to small changes in socket fit, and presence of the sensors does not disrupt the regular limb-socket interface. We did not consider placing the sensing elements on the actuators an appropriate strategy. The actuators are located at load-tolerant areas of the residual limb, thus sensing at those locations would not be expected to provide a clinically meaningful and sensitive measurement of socket fit. In the present study, the sensors used for auto adjustment were located at a posterior location off of the midline. It is possible that data from other sensors, for example that monitor distal limb superior-inferior motion (pistoning) or limb angulation in the sagittal plane, may be necessary, although results in the present study do not support such a need. These interpretations are preliminary, however, and need to undergo rigorous scientific testing on a larger group of participants.

### Control System Performance

The shape of the IAE curves over time, a rise and maximum followed by a decrease to a stable value, is typical for a properly functioning engineered control system. The range of end-of-bout IAE, from 0.001 to 0.034, corresponded to a median socket volume error of 0.001%-0.033% for the six participants tested here. For comparison, adding a sock sheath (0.2 mm thickness under stance phase loading) would change socket volume by an average of 0.97% for participants in the present study (range 0.80%-1.03%). Thus, the IAE error in this investigation would be expected to be acceptable in clinical application of the auto-adjusting socket.

The two bouts that showed the greatest increase in IAE over time were both from the only participant with a bulbous residual limb (participant #2). Unlike the other five people in the study, this participant found the relock protocol uncomfortable—drawing in the tether before closing the socket panels—because it tended to trap her soft tissue distally in the socket. Other participants, during preliminary investigations prior to this study and during the study itself, held the opposite opinion, preferring to draw in the tether first and then close the panels. The high IAE and discomfort stated by participant #2 suggest a need to investigate if a different auto-adjusting control strategy is necessary for people with much redundant soft tissue in their residual limb.

The oscillation of the SFM about the set point observed in some bouts may be eliminated by setting a threshold for execution of a socket size adjustment (known as a “hysteresis band” in control systems engineering). Because the peak-to-trough range of oscillation observed in the data collected in this study was typically less than 0.20 mm, we would expect that a hysteresis band and step size of 0.20 mm would be appropriate. It is possible that because of the different plant gains across participants, the threshold size adjustment may need to be tuned to each user, though rigorous scientific investigation would need to be conducted to test if this is clinically necessary.

We observed an interesting relationship between RSCR and percent limb fluid volume change in the posterior region from before to after the 1.0% socket size increase (Cycle 5-Cycle 4). The results suggest that matching the socket volume increase at the start of a new walking bout to the fluid volume increase experienced by the individual participant between bouts may improve comfort. In other words, the socket size should be adjusted so that the socket is still relatively snug on the residual limb and may even restrict its fluid volume increase. The four participants who experienced increases in percent fluid volume (cycle 5-cycle 4) of more than 1.0% (#1, #2. #3, and #6) all indicated that their socket felt too loose during cycle 5. Possibly the high fluid volume increase they experienced reduced their soft tissue compressive stiffness, made their limb-socket interface unstable, and caused this sensation. RSCR scores for the two participants who experienced lower percent fluid volume increase, #4 (−0.1% limb fluid volume change) and #5 (0.6%), were more favorable.

Possibly, participant #4 and #5's sockets were tight on their residual limb after the 1.0% socket size increase, helping to ensure a more stable interface. This interpretation is preliminary, based upon results from a small number of participants, and would need to be verified in more extensive clinical studies.

We note that by cycle 7 most participants returned to the limb fluid volume trajectory established before the +1.0% socket size intervention. Thus, while 10-min socket releases helped to reduce limb fluid volume loss, they may not have been long enough to promote a meaningful limb size increase such that a socket enlargement in preparation for further walking was warranted. Longer release durations, however, may show a benefit, though this hypothesis would need to be tested through rigorous scientific investigation.

The increase in peak-to-trough SFM (maximum swing phase SFM minus minimum stance phase SFM) from before to after the intervention (from cycle 4 to cycle 5) for participants who experienced a reduction in limb fluid volume over time (#1, #3, and #4) means that, as expected, these participants' limb pistoned more in the socket after the +1.0% relock socket size increase was executed than before it. The result points to the utility of SFM peak-to-trough data as an additional socket fit metric for use in future auto-adjusting socket systems.

A related prosthetic socket technology, electronic elevated vacuum (EV), applies a negative pressure between the socket and liner-to-draw residual limb soft tissues outward. Both EV and the auto-adjusting socket in the present study attempt to maintain limb volume within a narrow range over time, but they optimize different metrics to facilitate adjustment, and they use different actuators (vacuum pressure, panel displacement) to effect change. Results from the present study indicate that in-socket limb fluid volume response to automated control of socket size is immediate, while a different investigation reported in the literature showed that in-socket limb fluid volume response to automated control of EV is much slower. As research and development of both auto-adjusting sockets and EV sockets continue, it will be important to quantify their control system performance (i.e., capability to maintain a set point based on a socket fit metric), their capability to manage limb volume, and their benefit to clinical outcomes like residual limb health.

A limitation of the study design was that the system was tested on only six participants. This number of participants was considered acceptable because the objective of the study was to warrant at-home testing with a large number of participants.

The time between walking bouts in this study was short, approximately 10 min. A more challenging situation, expected to be encountered during at-home use, is when there are much longer time periods of sitting, standing, and weight-shifting between walking bouts, that is, between automatic panel position changes. In these cases, a more substantial change in the residual limb may occur and the SFM at the outset of the next walk may be much different than the set point. If the difference is too great, then the residual limb may not be able to adjust size quickly, risking control system instability and necessitating a modification in the control system strategy.

The auto-adjusting socket used in this study was heavier than a normal socket. The instrumentation on the socket added a median of 885 g to the traditional socket weight (median 589 g). The weight may have affected participant RSCR scores late in the session because of the accumulated effect of a greater pull on limb soft tissues compared with users' traditional sockets. We would not expect the added weight to have affected control system performance since the auto-adjusting socket adapts to a change in socket fit. A reduced size frame and motor and replacement of the control system components with a custom electronics board could easily be created to reduce the weight since from experience in the present study the displacement range and resolution needs of the auto-adjusting socket are now better specified. For the revised system, we would expect the weight difference compared to a traditional prosthesis to be comparable to the difference between a powered ankle and traditional ankle prosthesis. The LabVIEW VI instability issue during some bouts in the present study should be resolved using an on-board microprocessor dedicated to auto-adjustment instead of the Lab VIEW software package.

### D. An Instrumented Printed Insert for Continuous Monitoring of Distal Limb Motion in Suction and Elevated Vacuum Sockets

### Materials and Methods

An inductive sensing modality was used to monitor residual limb motion in the socket. Antenna coils printed on a flex-circuit (polyimide) and placed in the socket are powered using an inductive sensing chip. A trace amount of iron powder embedded in the participant's liner serves as the antenna's target. When the antenna and circuitry are powered, the presence of the magnetically permeable iron local to the antenna reinforces the inductor and lowered the sensor's oscillation frequency in a distance-dependent manner. The change in frequency measured by the inductive sensing chip is a sensitive measure of distance between the antenna and target.

### Insert Design and Assembly

A custom 3D printed plastic insert was designed to hold two antennae (FIG. 13). An insert was used rather than adhering the antennae directly to the inside of the socket because in prior research we found that the antennae were damaged within 1-2 weeks from the mechanical stress applied by the residual limb. The insert matched the shape of the distal portion of the socket. Similar to the full socket inserts we de-signed in previous work, recesses and channels were cut out of the external surface. The recesses and channels held the custom inductive sensor antennae and leadwires. The insert was designed as thin as possible because the long-term objective was a technology to be placed into an existing socket with minimal distortion to socket shape or volume. This design strategy would allow clinical evaluation of an existing socket suspected of vacuum loss. In the present study, however, to ensure that the shape of the socket was not a variable in the study, we fabricated research prostheses where we adjusted the socket shape to accommodate the insert thickness. The insert was affixed to the inside of the socket using double-sided tape, and the cabling was routed through a small hole in the socket wall that was sealed to prevent leaks.

To design the insert, we used a coordinate measurement machine (FaroArm Platinum, FARO Technologies) (accuracy setting 0.02 mm) to digitize the shape of the participant's regular socket. The socket brim was also digitized so that the anterior and posterior directions were easily identified when placing the insert into the socket. A surface was made from the point cloud data using a computer-aided design software package (Geomagic, Design X, 3D Systems). This surface was projected radially outward (perpendicular to the surface) 1.2 to 1.8 mm to create a solid model of uniform thickness. The amount depended on the desired insert thickness. Different thickness and materials were explored to achieve a mechanically durable insert that did not compress the sensors during use.

Parametric modeling software (Inventor, Autodesk) was used to create recesses and channels in the external surface of the insert to hold two custom-designed flexible coil antennae (32.0-mm diameter, 0.15-mm thickness) (FIG. 13). Material from the center of the antenna was removed to enhance the flexibility so that the insert well conformed to the socket shape (FIG. 2A). The flex-circuit antenna was designed and fabricated to include a 16 mm×10 mm “tail” with two pairs of exposed solder tabs near the ends of the trace. The solder tabs were used to hold surface mount electronics (0.22 μF capacitor: 10 kΩ thermistor) and connected the leadwires to the inductive sensing chip. Each antenna was positioned within a 0.8-mm deep spherical ring-shaped recess in the insert and held in place along the inside edge using hot melt adhesive (3779, 3M). The center island of the recess, which projected through the center of the antenna, provided structural support to avoid mechanical pressure on the antenna. Recesses 0.8-mm deep were also made for the antenna tail, and a through hole was made in the insert at the location the two tails met, which was right over a 9.0-mm diameter hole (described below) where the leadwires exited through the socket wall. This design created sufficient space to allow 1.0-mm diameter leadwire to be used to connect from the solder tabs through the socket wall to a signal conditioner/data logger fastened to the pylon of the prosthesis. A data logger from our previous work was used. A thin piece of ferrite (Würth Elektronik, Niedernhall, Hohenlohe) (0.33-mm thickness) was adhered over the outside of the antenna to reduce radio frequency interference and to provide electromagnetic interference shielding from the carbon fiber socket A series of slits were cut in the ferrite to improve flexibility (FIG. 2C). A bottom view of a fabricated insert ready for attachment of two antennae and leadwires is shown in FIG. 13.

Three insert materials were considered—Veroclear (Stratsys): Nylon PA12 40% glass filled; and PerFORM (DSM Functional Materials, Somos Materials Group). Two thicknesses of the PerFORM were tested—1.8 mm and 1.2 mm.

Additional steps were taken to prepare the insert and socket. Hot melt was placed over the surface mount capacitor and thermistor. A 7.0-mm hole was drilled in the center distal end of the insert and socket to allow air to pass to the suction valve or vacuum componentry hardware immediately beneath the socket. Hot melt was placed to cover the connection between the leadwires and the antenna tail. A 9.0-mm diameter hole (for the leadwires) was drilled through the socket wall underneath the cutout (FIG. 14).

### Installation in the Prosthetic Socket

Sockets were fabricated using materials commonly used in clinical practice. The socket includes 4 layers of resin/carbon fiber and 2 layers of Nyglass.

In preparation for installation, the external surface of the insert that was not covered by antennae or leadwires was covered with double-sided adhesive tape (SpeedTape, FastCap) (0.05-mm thickness). The insert was carefully positioned inside the socket while pulling the leadwires through the 9.0-mm hole. Marks previously inked on the anterior and posterior aspects of the insert and socket during test assembly facilitated alignment. An abrasion-resistant expandable sleeve was used to protect the bundle of leadwires exiting the socket. The 7 0-mm and 9.0-mm holes were sealed with epoxy (PLU Series Composite 1, FabTech). A vacuum bag was placed over the socket to test for leaks. If the socket held at least 25 inHg for at least a 30 s duration, the seal was considered acceptable.

### Calibration

Calibration was performed using the liner to be worn by the participant. For this study, liners for research purposes with a trace amount of iron powder embedded in the elastomer beneath its surface were purchased (Alpha Classic, Willow Wood). We used a procedure similar to that in our prior work calibrating sensors in pin-lock sockets. First, the anterior and posterior surfaces of the liner over the sensor locations were calibrated in a benchtop test jig that allowed an antenna to be moved at incremental distances perpendicular to the liner. This procedure generated the shape of the calibration curve. To establish the zero reference position (liner flush with the insert), the liner was placed in the participant's research socket while vacuum pressure was applied through the 7.0-mm hole in the distal end of the socket. A silicone balloon in the shape of a residual limb was placed inside a sock, and that assembly was placed inside the liner. A gaitor sleeve was slid into position, and the proximal end of the liner was folded over it. A gaitor was used because during preliminary take-home tests, we found that the sealing sleeve was prone to mechanical damage from the socket brim. The gaitor protected the sealing sleeve. The sealing sleeve was pulled into place to seal both the proximal and distal ends of the socket. The balloon was held at a pressure of approximately 3.4 kPa, and the sensor data acquisition system was started. A 3.4 kPa pressure was used because this was sufficiently high to push the liner against the socket wall but not so high that the balloon was forced out the top of the socket. Five vacuum pressures were applied for 45 s each (5, 10, 15, 20, 25 inHg). Vacuum pressure was returned to 0 inHg for 15 s after each setting. Sensor data at the 10 inHg setting was used as the reference (0 distance) for calibration because it best achieved the objective of the liner flush with the insert without distortion of the liner shape.

### Study Protocol

The participant visited the lab three times. At the first visit, the research prosthetist conducted a clinical evaluation to determine if inclusion criteria were met. A full inspection of the person's residual limb was conducted. Demographic information, etiology, medical history, presence of co-morbidities, regularly conducted activities, smoking status, and the design of the currently used prosthesis were recorded while the participant's socket was scanned with the high-resolution coordinate measurement machine. Body mass index (BMI) was calculated using the formula for people with transtibial limb loss.

A carbon-fiber/resin socket duplicate in shape to the participant's regular socket was fabricated as described above and the instrumented insert installed. Care was taken to ensure that tight seals were achieved at the suction or vacuum port and at the sensor cable exit from the socket. Participants wore the socket for several hours to ensure that the insert adhesive compressed to a consistent thickness, as learned in our prior research using socket inserts in clinical studies.

During the second visit, participants conducted an in-lab structured protocol wearing the research prosthesis. Participants donned the research socket, and the re-search prosthetist made any necessary adjustment to ensure a proper fit. This was the optimal sock thickness and was termed sock opt. Sensor data collection was initiated. Participants executed three activity cycles four times for a total of twelve activity cycles. Each cycle included: standing (10) s); walking on a treadmill (2 min); standing (10 s); and sitting (2 min). After the first three activity cycles (3 of the 12 cycles). participants doffed the research socket and increased their sock ply by adding a sock or changing a current sock to a thicker ply. Participants chose among 1, 2, 3, and 5 ply socks (1-2 ply: 95.5% Tertra-Channel polyester, 4.5% Lycra; 3 ply: 70% wool, 30% tet-ra-channel polyester; 5-ply: 60% wool, 40% Tertra-Channel polyester, Royal Knit, Inc.). They conducted three activity cycles at this sock ply. Participants sat and doffed their prosthesis, increased their sock play again, and conducted three additional activity cycles at this sock ply. Participants sat and doffed, returned to their starting configuration (sock opt), which was a low ply sock or no sock, and conducted three additional activity cycles. After the twelve activity cycles were completed, participants sat in a chair and the researchers prepared them for the take-home part of the study. Participants were asked to wear the research prosthesis at home for at least 3 d, conducting their daily routine and monitoring their comfort via notes or verbal correspondence with the research team. They returned to the lab for their third visit after approximately 1 week to return the research prosthesis. Participants were encouraged to provide comments on their experience during take-home use wearing the research prosthesis.

Collected data were thermally compensated and converted to units of distance (mm) using the calibration data. For the in-lab structured protocol data, only the walking sections were analyzed. The maxima (which occurred during swing phase) and minima (which occurred during stance phase) for the last ten steps in each cycle were extracted and medians calculated. Anterior and posterior sensor results were calculated separately. The last ten steps were used because in some cycles we observed a change in sensed distance during the initial steps, in part because the vacuum pressure had yet to stabilize.

Take-home data were processed to identify the start and end of prosthesis use each day and to classify user activity as walking, weight shift (standing or sitting), stationary (standing or sitting), or doffed (partial or full). Walks were further categorized into either bouts (>5 steps) or low commotion (2-4 steps). The percentage time for each prosthesis use across all prosthesis day durations was calculated, and the number of steps per day was computed. The median peaks, valleys, and amplitudes (range) were calculated for all steps within each hour of wear and plotted. To visually investigate trends in the data over time, we plotted the data for each step and a 21-point moving average. Histograms were generated to characterize the distribution of the peaks, valleys, and amplitudes in the steps for each day of use.

### Discussion

Monitoring distal limb motion in sockets that use suction and elevated vacuum suspension could help improve performance of lower-limb prostheses. Research studies using this technology may characterize how variables under practitioner and patient control (e.g., vacuum pressure, sealing sleeve material) or prosthesis use variables (e.g., type of activity, duration of use) affect a meaningful outcome variable, limb motion. Results may help guide prosthesis prescription, and they may stimulate development in the prosthetics industry of more effective and reliable limb suspension products. Continuous distal limb sensing has the potential to be part of an auto-adjustable socket that changes both socket size and vacuum pressure to maintain proper suspension.

We chose to monitor cyclic vertical motion at a distal location in the socket since the suction valve or elevated vacuum pump port is typically located at this position. This location is clinically meaningful to monitor for a loss of vacuum pressure. Further, we sought to position the sensing system in such a way that it introduced minimal disruption to the normal prosthesis, avoiding introducing additional hardware on the outside of the socket.

Inductive sensing offers advantages over other techniques reported in the literature to monitor distal limb motion in transtibial prosthesis users. An optical sensing unit positioned underneath the socket to track distal limb motion demonstrated less cyclic vertical motion when a participant used a supracondylar strap compared with no strap, but it required a hole to be cut through the user's Pelite™ liner to take the measurement. Radiographic methods implemented to track bone position while participants transitioned to weight-bearing showed less tibia motion under elevated vacuum compared with passive suction, locking pin, patellar tendon bearing, and supracondylar suspension, but the method required large equipment and exposed participants to radiation. Both techniques would be difficult to implement in a take-home socket. Optical techniques to monitor motion between the liner and side of the socket have been developed, but the need for either a clear socket material or a small hole through the wall make these methods difficult to implement at a distal location. A dipole magnet disk affixed to the liner and tracked with a sensor mounted outside of the socket achieved sub-millimeter resolution, but the technique would need to be modified to measure perpendicular instead of tangential motion for use in the present application. A distal pressure sensor was shown to distinguish sock addition and removal during a structured protocol, though no suction or elevated vacuum sockets were tested. Thin piezoresistive pressure sensors placed at the limb-socket interface did not assess limb cyclic motion since loss of socket contact causes them to sense zero pressure or vacuum pressure during swing phase.

In-lab test results in the present study demonstrated that all participants moved further away from the socket at both the anterior and posterior sensor sites when socks were added and moved closer when socks were removed. Given the short time course of this test, this result is consistent with expectation and verified proper sensor performance. Sock changes distributed over a longer time period, for example during regular at-home use, might not show such consistent results because the residual limb may change volume over time, and limb-socket alignment may change. The complexity of interface stress distribution changes over time has been studied.

When sock ply was reduced (cycles 10 to 12) the people who regularly used elevated vacuum sockets returned closer to baseline than the participant who regularly used a suction socket. It is possible that the soft tissues of the regular elevated vacuum users had adapted differently than suction users. Possibly, the continually applied higher vacuum pressure caused residual limb tissues to change their volume quickly in response to a change in interface pressure. This interpretation is conjecture and would need to be tested through rigorous scientific investigation. Residual limb tissue adaptation to regular use of elevated vacuum has been discussed.

The result that cyclic vertical motion increased when socks were added to a properly fitting socket is consistent with clinical experience. Socks may increase the slip between the liner and the socket, and may also introduce air, a compressible medium, to that interface. Air presence may increase cyclic vertical motion because the air compresses during stance phase and expands during swing phase. Participants 3 and 4, who wore no socks for the optimal sock (sock opt) condition during the in-lab test. showed less limb cyclic vertical motion for the sock opt condition than participants 1 and 2, who did wear socks for the sock opt condition. During take-home use, participant 3, the only person who did not wear socks, experienced lower and more consistent limb motion amplitude than the other participants. The results suggest that adding socks negates part of the intent of suction, to reduce limb motion, and should be avoided. As described in comments collected after completing take-home use. participants added socks because they usually used a sock (participant 1) or because the extra weight of the research prosthesis compared to their traditional caused them to feel that sock addition was necessary (participants 2 and 4). The results from the present study provide incentive for prosthetics researchers and the industry to investigate sockets that allow size change while maintaining vacuum. Using adjustable-size sockets or providing participants with various thickness custom plastic inserts may accomplish this design objective. Results from a benchtop model of elevated vacuum suggest that the effect of vacuum pressure on the residual limb is primarily determined by air gap distance.

Results from this study do not support the hypothesis that the effectiveness of suspension in suction sockets is reflected as a bimodal distribution of motion. i.e., that there is one amplitude reflecting good suspension and another amplitude reflecting slip. It is clear from the tight distribution of the data during the in-lab test compared with that during take-home use that there may be considerable fluctuation in at home environments for how hard participants push into their socket during stance phase and how forceful they pull out during swing phase. Inspection of the histograms in the present study suggests that it is the peaks during swing phase that caused the high variability since their distributions were wider than those of the valleys. Participant 3 showed a bimodal distribution for the valleys on day 6, because of either lower weight bearing or less swing pull out during some of his bouts of low commotion activity. The result is consistent with moving about in a workshop, which this person reported as a common activity.

The insert materials used in this study were much stiffer and more durable than the larger thicker instrumented inserts we used in prior studies. The PerFORM material, with its low elongation at break (1.1%), did not crack, demonstrating it as the best choice of the materials tested here. The thinnest insert that we could fabricate was 1.2 mm, which is too thick to add to a participant's regular socket because the reduced socket volume may cause discomfort. We believe that the inserts are more appropriate to put into new sockets, enlarging the distal end in the computer-aided design file as done in this study or by placing a dummy insert over the positive mold during the layup. Care must be taken to smooth the insert edge so that it does not cause irritation. An alternative design is to eliminate the insert and place the sensor antennae directly in the layup during fabrication, a procedure that we have described. Placement within 3D printed sockets may be possible, once the industry advances and printed materials sufficiently strong for this application are available

The most meaningful limitation in this study that affected socket use in take-home testing was the weight of the research prosthesis compared with participants' regular prosthesis. This was not a limitation of the inserts, but instead the lack of available blade prostheses to laminate to participants' research sockets. A comparatively heavy pylon and energy storage and return foot were used instead. An interesting next step would be to match participants' regular componentry in an investigational prosthesis to achieve comparable weight to the traditional, and to conduct testing for weeks instead of days. One participant felt that the research liner, which had a trace amount of iron powder in the elastomer next to the fabric backing, was stiffer and not as comfortable as his regular liner. However, the research liner was a different product than the participant's normal liner, which may have contributed to this statement.

Because only distal locations were monitored in the present study, we were not able to distinguish standing from sitting nor a partially doffed from a fully doffed prosthesis in our prosthesis use characterization. In our prior research, we effectively used four sensors to accomplish these distinctions, two distal and two proximal. If the sit-stand distinction had been available, it would have been possible to determine if there was a greater increase in cyclic vertical motion after sitting than after standing, suggesting a greater loss of suction during sitting. The addition of a vacuum pressure sensor during data collection would allow evaluation of the relationship between vacuum pressure and displacement. A linear relationship was found by Gerschutz et al. using a structured protocol in the lab. It would also be interesting to simultaneously monitor motion on the side of the socket, as pursued by several research groups. to gain insight into how the magnitude of motion changes from the proximal to the distal end of the socket.

### Conclusion

The designed insert performed well in that it accurately measured with high resolution distal limb position and motion in people using transtibial prostheses with suction suspension. Potentially, this technology can be used as an effective tool to facilitate improvement in suction and elevated vacuum technology.

Data collected from four participants suggest that research investigation into cyclic limb motion for sock presence v. absence should be considered, as should the influence of bodily position between bouts of walking. These variables may have an important influence on suspension.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

1. A prosthetic socket comprising:
a first layer defining a cavity;
a second layer positioned over the first layer;
a plurality of sensors positioned between the first layer and the second layer, wherein each of the plurality of sensors are configured to measure a distance from a residual limb positioned within the cavity; and
a communication interface in communication with each of the plurality of sensors.

2. The prosthetic socket of claim 1, wherein each of the plurality of sensors is an inductive sensor.

3. The prosthetic socket of claim 1,
wherein the first layer further comprises an electrically insulating webbing material and a curable matrix.

4. The prosthetic socket of claim 1,
wherein the second layer further comprises carbon fiber webbing and a curable matrix.

5. The prosthetic socket of claim 4, wherein each of the plurality of sensors has a ground wire threaded into carbon fiber layer.

6. The prosthetic socket of claim 1,
wherein each of the plurality of sensors further comprises:
an antenna;
a thermistor; and
a capacitor
wherein the thermistor and the capacitor are co-located with the antenna.

7. The prosthetic socket of claim 1,
wherein each of the plurality of sensors further comprises:
a flexible antenna having a front side and a back side;
an electrically insulating layer having a front side and a back side, wherein the front side is fixed to the back side of the flexible antenna; and
a ferrite outer layer fixed to the back side of the electrically insulating layer.

8. The prosthetic socket of claim 2, wherein the inductive sensor further comprises a looped antenna fixed to a flexible substrate, and wherein the flexible substrate includes a cut out substantially in the center of a circle defined by the looped antenna.

9. The prosthetic socket of claim 8, wherein the sensor layers have an outer perimeter edge and an inner perimeter edge defined by the cut out, and wherein any of the sensor layers include alternating radial slit cuts from the outer perimeter toward the looped antenna and from the inner perimeter toward the looped antenna.

10. (canceled).

11. The prosthetic socket of claim 1, wherein the plurality of sensors are configured to measure socket fit during prosthesis use, and wherein:
a first sensor is positioned in a posterior distal region of the socket at a first distance from a midline of the socket;
a second sensor is positioned in a posterior distal region of the socket at the first distance from the midline opposite the first sensor;
a third sensor positioned in an anterior medial proximal region of the socket; and
a fourth sensor in an anterior lateral proximal region of the socket.

12. (canceled).

13. The prosthetic socket of claim 1, wherein the plurality of sensors is configured to measure socket fit to provide feedback for adjusting socket fit, and wherein:
a first sensor is positioned at an anterior distal end of the socket;
a second sensor is positioned at an anterior proximal end of the socket;
a third sensor is positioned between sensor one and sensor two on an anterior side of the socket;
a fourth sensor is positioned at a medial mid-limb location on a posterior side of the socket;
a fifth sensor is positioned at a lateral mid-limb location on a posterior side of the socket; and
a sixth sensor is positioned at a posterior distal end of the socket.

14. The prosthetic socket of claim 13, further comprising:
a controller configured to receive a set of signals from the plurality of sensors;
an adjustable panel in the socket configured to loosen or tighten socket fit with adjustment;
an electric motor configured to move the adjustable panel; and
a power source,
wherein the controller is configured to actuate the electric motor based on, at least in part, received signals from the plurality of sensors.

15. The prosthetic socket of claim 14, wherein
the controller actuates the adjustable panel to loosen in response to the set of signals from the plurality of sensors reporting increasing limb volume; and
the controller actuates the adjustable panel to tighten in response to the set of signals from the plurality of sensors reporting decreasing limb volume,
wherein the controller sets a comfortable fit distance from the plurality of sensors upon donning the socket, and the controller works to maintain the comfortable fit distance.

16. The prosthetic socket of claim 14, wherein the set of received signals from the plurality of sensors classify one of the following prosthetic use conditions:
stationary sitting;
stationary standing;
sit-to-stand transitioning;
walking;
weight-shifting sitting;
weight-shifting standing
partial doffing; and
non-use.

17. A method of making an instrumented prosthetic socket comprising:
forming an inner layer of material over a mold;
placing a plurality of sensors on an outer surface of the inner layer; and
forming an outer layer of material over the sensors and inner layer.

18. The method of claim 17, wherein the inner layer comprises an electrically insulating webbing and a curing matrix.

19. The method of claim 17, wherein placing a plurality of sensors includes placing wiring from the sensors to a controller.

20. A prosthetic socket comprising:
a socket configured to receive a limb, wherein the socket further comprises:
a first layer, and
a second layer; and
a plurality of sensor arrays between the first layer and second layer of the socket,
wherein each sensor array of the plurality of sensor arrays comprises at least three sensors, and
wherein each sensor array is configured to measure a three-dimensional position from the sensor array's position in the socket to a single target material.

21. The prosthetic socket of claim 20, wherein each sensor is configured to provide to a controller a distance data to a sensor target and wherein the controller trilateralizes the distance data from each sensor array to provide a three dimensional position data of the sensor target relative to the sensor array.

22. The prosthetic socket of claim 21, wherein a lookup table converts the distance data from each of the at least three sensors to a three dimensional position data.
